(19) United States
(12) Patent Application Publication
(10) Pub. No.: US 2023/0248249 A1
(43) Pub. Date: Aug. 10, 2023

(54) IMPLANTABLE PRESSURE SENSOR PACKAGING

(71) Applicant: EDWARDS LIFESCIENCES CORPORATION, Irvine, CA (US)

(72) Inventors: Yaron Keidar, Kiryat Ono (IL); Mark Simon Vreeke, Aliso Viejo, CA (US); Ming H. Wu, Tustin, CA (US); Alexander H. Siemons, Yorba Linda, CA (US)

(21) Appl. No.: 18/174,155

(22) Filed: Feb. 24, 2023

Related U.S. Application Data
(63) Continuation of application No. PCT/US21/45750, filed on Aug. 12, 2021.
(60) Provisional application No. 63/069,907, filed on Aug. 25, 2020.

(51) Int. Cl. A61B 5/0215; A61B 5/00

(52) U.S. Cl. CPC A61B 5/0215; A61B 5/6869; A61B 5/6885

(57) ABSTRACT

An implantable sensor device includes a sensor-support substrate, a microelectromechanical systems (MEMS) pressure sensor device mounted to the sensor-support substrate, a transduction medium applied over the pressure sensor device, and a biocompatibility layer applied over the transduction medium

### RELATED APPLICATIONS

This application claims the benefit of PCT/US2021/045750 filed Aug. 12, 2021, which claims priority to U.S. Provisional Patent Application Ser. No. 63/069,907, filed on Aug. 25, 2020 and entitled IMPLANTABLE PRESSURE SENSOR PACKAGING, the complete disclosures of which are hereby incorporated by reference in their entireties.

### BACKGROUND

### Field

The present disclosure generally relates to the field of medical implant devices.

### Description of Related Art

Various medical procedures involve the implantation of a medical implant devices within the body, such as within various chambers and anatomy of the heart. Sensor devices can be used to measure certain physiological parameters associated with such anatomy, such as fluid pressure, which can have an impact on patient health prospects.

### SUMMARY

Described herein are methods, systems, and devices that facilitate the implantation and/or maintenance of implantable pressure sensors in the body. In particular, various pressure sensor packaging solutions are disclosed that provide advantageous pressure-transducing and biocompatibility-enhancing characteristics.

In some implementations, the present disclosure relates to an implantable sensor device comprising a sensor-support substrate, a microelectromechanical systems (MEMS) pressure sensor device mounted to the sensor-support substrate, a transduction medium applied over the pressure sensor device, and a biocompatibility layer applied over the transduction medium.

The implantable sensor device may further comprise one or more bondwires electrically coupled to the pressure sensor device. For example, the transduction medium may cover at least a portion of the one or more bondwires. The sensor-support substrate can include one or more through-holes through which at least one of the one or more bondwires pass to a backside of the sensor-support substrate.

The transduction medium can comprise any one or more of the group consisting of parylene, silicone, and epoxy.

The transduction medium can have a non-conformal top surface or can have a conformal surface conforming to a form of the pressure sensor device.

In some embodiments, the biocompatibility layer comprises a metal film.

The implantable sensor device may further comprise an oxide layer formed on a surface of the biocompatibility layer. An organic film may be bonded to the oxide layer. For example, the organic film may be covalently bonded to the oxide layer. In some embodiments, the organic film comprises at least one of polyethylene glycol, a long-chain organic acid, a protein, or a carbohydrate.

In some embodiments, the sensor-support substrate comprises metal.

The pressure sensor device, the transduction medium, and/or biocompatibility layer are disposed at least partially within sidewalls that are mechanically coupled to the sensor-support substrate.

In some implementations, the present disclosure relates to a method of packaging a pressure sensor device. The method comprises providing a microelectromechanical systems (MEMS) pressure sensor device mounted to a sensor-support substrate, applying a transduction medium over the pressure sensor device, and applying a biocompatibility layer over the transduction medium.

Applying the transduction medium can comprise covering the pressure sensor device and at least a portion of one or more bondwires electrically coupled to the pressure sensor device with the transduction medium.

The transduction medium may comprise one or more of parylene, silicone and/or epoxy.

The transduction medium can have a non-conformal top surface or can have a conformal top surface.

Applying the transduction medium can comprise forming a conformal layer of the transduction medium over the pressure sensor device and at least a portion of the sensor-support substrate.

Applying the biocompatibility layer can comprise sputtering a titanium film onto the transduction medium.

The method can further comprise forming an oxide layer on a surface of the biocompatibility layer. The method can further comprise bonding an organic film to the oxide layer.

In some implementations, the present disclosure relates to a pressure sensor assembly comprising a metal can structure including a base and one or more sidewalls, a microelectromechanical systems (MEMS) pressure sensor device mounted to the base of the metal can structure, a printed circuit board electrically coupled to the pressure sensor device via one or more through-holes in the base of the metal can structure, a coil antenna electrically coupled to the printed circuit board, a rigid tube encapsulating at least a portion of the printed circuit board and the coil antenna, the rigid tube being mechanically secured to the metal can structure, a transduction medium applied over the pressure sensor device within the one or more sidewalk of the metal can structure, and a biocompatibility layer applied over the transduction medium.

The transduction medium can comprise one or more of parylene, and/or epoxy. The transduction medium can have a non-conformal top surface or can have a conformal surface conforming to forms of the pressure sensor device.

In some embodiments, the biocompatibility layer comprises a metal film.

The pressure sensor assembly can further comprise an oxide layer formed on a surface of the biocompatibility layer. An organic film can be bonded to the oxide layer.

In some implementations, the present disclosure relates to a pressure sensor assembly comprising a printed circuit board, a wireless transmitter electrically coupled to the printed circuit board, a rigid tube encapsulating at least a portion of the printed circuit board and the wireless transmitter, the rigid tube having first and second ends, a microelectromechanical systems (MEMS) pressure sensor device mounted to an end portion of the printed circuit board that extends axially beyond the first end of the rigid tube, a transduction medium that covers the printed circuit board, the wireless transmitter, and the pressure sensor device, the transduction medium filling the rigid tube and projecting axially beyond the first end of the rigid tube over the end portion of the printed circuit board, and a biocompatibility layer applied over the first and second ends of the rigid tube and over portions of the transduction medium associated with the first and second ends of the rigid tube, respectively.

The transduction medium can comprise one or more of parylene, and/or epoxy.

The pressure sensor assembly may further comprise a polymer layer applied over at least a portion of the biocompatibility layer.

In some embodiments, the biocompatibility layer comprises alternating layers of polymer and metal. For example, the alternating layers of polymer and metal can comprise at least two layers of metal and at least two layers of polymer.

The pressure sensor assembly may further comprise an oxide layer is formed on a surface of the biocompatibility layer. The pressure sensor assembly may further comprise an organic film bonded to the oxide layer.

In some implementations, the present disclosure relates to an implantable sensor device comprising a sensor-support substrate, a microelectromechanical systems (MEMS) pressure sensor device mounted to the sensor-support substrate, a transduction medium applied over the pressure sensor device, and a biocompatibility layer applied over the transduction medium, the biocompatibility layer comprising alternating sublayers of metal film and polymer film.

The alternating sublayers of metal film and polymer film can comprise at least two sublayers of metal film and at least two sublayers of polymer film. For example, the alternating sublayers of metal film and polymer film comprise at least ten film sublayers. In some embodiments, the alternating sublayers of metal film and polymer film comprise at least twelve film sublayers.

At least some sublayers of the biocompatibility layer may have a thickness of about 1 pm or less.

In some embodiments, the biocompatibility layer has a thickness of about 10 μm or less.

In some embodiments, bottom and top sublayers of the biocompatibility layer are metal film sublayers.

For purposes of summarizing the disclosure, certain aspects, advantages and novel features have been described. It is to be understood that not necessarily all such advantages may be achieved in accordance with any particular embodiment. Thus, the disclosed embodiments may be carried out in a manner that achieves or optimizes one advantage or group of advantages as taught herein without necessarily achieving other advantages as may be taught or suggested herein.

### BRIEF DESCRIPTION OF THE DRAWINGS

Various embodiments are depicted in the accompanying drawings for illustrative purposes and should in no way be interpreted as limiting the scope of the inventions. In addition, various features of different disclosed embodiments can be combined to form additional embodiments, which are part of this disclosure. Throughout the drawings, reference numbers may be reused to indicate correspondence between reference elements.

FIG. 1 illustrates an example representation of a human heart in accordance with one or more embodiments.

FIG. 2 illustrates example pressure waveforms associated with various chambers and vessels of the heart according to one or more embodiments.

FIG. 3 illustrates a graph showing left atrial pressure ranges.

FIG. 4A is a side view of a piezoresistive MEMS pressure sensor device in accordance with one or more embodiments.

FIG. 4B is a side view of the piezoresistive MEMS pressure sensor of FIG. 4A, wherein a diaphragm of the sensor is deflected in accordance with one or more embodiments.

FIG. 4C shows a plan view of the diaphragm of the sensor device shown in FIGS. 4A and 4B in accordance with one or more embodiments.

FIG. 5A is a side view of a capacitive MEMS pressure sensor device in accordance with one or more embodiments.

FIG. 5B is a side view of the capacitive MEMS pressure sensor of FIG. 5A, wherein a diaphragm of the sensor is deflected in accordance with one or more embodiments.

FIG. 6 is a block diagram representing an implant device in accordance with one or more embodiments.

FIG. 7 is a block diagram representing a system for monitoring one or more physiological parameters associated with a patient according to one or more embodiments.

FIG. 8 is a back and side perspective view of a sensor implant device in accordance with one or more embodiments.

FIG. 9 shows a front and side perspective view of the sensor implant device of FIG. 8 in accordance with one or more embodiments.

FIG. 10 provides an exploded view of the sensor can package of the sensor implant device of FIGS. 8 and 9 in accordance with one or more embodiments.

FIG. 11 shows a cross-sectional view of the sensor implant device 100 shown in FIGS. 8-10 in accordance with one or more embodiments.

FIG. 12 illustrates a sensor implant device having sensor-support strut or arm in accordance with one or more embodiments.

FIG. 13 is a side view of a pressure sensor package in accordance with one or more embodiments.

FIG. 14 is a side view of a pressure sensor package in accordance with one or more embodiments.

FIG. 15 is a side view of a pressure sensor package in accordance with one or more embodiments.

FIG. 16 is a side view of a pressure sensor package in accordance with one or more embodiments.

FIG. 17 is a side view of a pressure sensor package in accordance with one or more embodiments.

FIG. 18 is a side view of a pressure sensor package in accordance with one or more embodiments.

FIG. 19 is a side view of a pressure sensor package in accordance with one or more embodiments.

FIG. 20 is a side view of a pressure sensor package in accordance with one or more embodiments.

FIGS. 21-1 21-4 are a flow diagram illustrating a process for packaging a sensor implant device in accordance with one or more embodiments.

FIGS. 22-1-22-4 provide images of pressure sensor packaging corresponding to operations of the process of FIGS. 21-1-21-4 according to one or more embodiments.

FIG. 23 is a side cross-sectional view of a packaged pressure sensor device in accordance with one or more embodiments.

FIG. 24 is a front and side perspective view of the packaged pressure sensor device of FIG. 23 in accordance with one or more embodiments.

FIG. 25 is a side cross-sectional view of a packaged pressure sensor device in accordance with one or more embodiments.

FIG. 26 is a side cross-sectional view of a packaged pressure sensor device in accordance with one or more embodiments.

FIG. 27 is a side cross-sectional view of a packaged pressure sensor device in accordance with one or more embodiments.

FIG. 28 illustrates various access paths through which access to a target cardiac anatomy may be achieved in accordance with one or more embodiments.

### DETAILED DESCRIPTION

The headings provided herein are for convenience only and do not necessarily affect the scope or meaning of the claimed invention.

Although certain preferred embodiments and examples are disclosed below, it should be understood that the inventive subject matter extends beyond the specifically disclosed embodiments to other alternative embodiments and/or uses and to modifications and equivalents thereof. Thus, the scope of the claims that may arise herefrom is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Certain standard anatomical terms of location are used herein to refer to the anatomy of animals, and namely humans, with respect to various embodiments. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa. It should be understood that spatially relative terms, including those listed above, may be understood relative to a respective illustrated orientation of a referenced figure.

The present disclosure relates to systems, devices, and methods for packaging devices configured for telemetric monitoring of one or more physiological parameters of a patient (e.g., blood pressure). Such pressure monitoring may be performed using cardiac implant devices having integrated pressure sensors and/or associated components. For example, in some implementations, the present disclosure relates to cardiac shunts and/or other cardiac implant devices that incorporate or are associated with pressure sensors or other sensor devices packaged for long-term implantation in the cardiac environment. The term “associated with” is used herein according to its broad and ordinary meaning. For example, where a first feature, element, component, device, or member is described as being “associated with” a second feature, element, component, device, or member, such description should be understood as indicating that the first feature, element, component, device, or member is physically coupled, attached, or connected to, integrated with, embedded at least partially within, or otherwise physically related to the second feature, element, component, device, or member, whether directly or indirectly.

As described in detail below, implantable pressure sensors can be used to measure pressure levels in various conduits and chambers of body, such as in the various chambers of the heart. However, due to the accessibility and environmental conditions typically associated with the conduits/chambers of the heart and/or other potential sensor implant locations within a patient, only certain types of sensors and sensor packagings may be suitable for implantation for a given application. Embodiments of the present disclosure relate to the packaging of pressure sensor implant devices including certain electronics and telemetry features to allow for data and/or power communication wirelessly between the implanted sensor devices and one or more devices or systems external to the patient.

### Cardiac Physiology

Certain embodiments are disclosed herein in the context of cardiac implant devices. However, although certain principles disclosed herein may be particularly applicable to the anatomy of the heart, it should be understood that sensor implant devices in accordance with the present disclosure may be implanted in, or configured for implantation in, any suitable or desirable anatomy.

The anatomy of the heart is described below to assist in the understanding of certain inventive concepts disclosed herein. In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to blood vessels (e.g., pulmonary, aorta, etc.). The contraction of the various heart muscles may be prompted by signals generated by the electrical system of the heart, which is discussed in detail below.

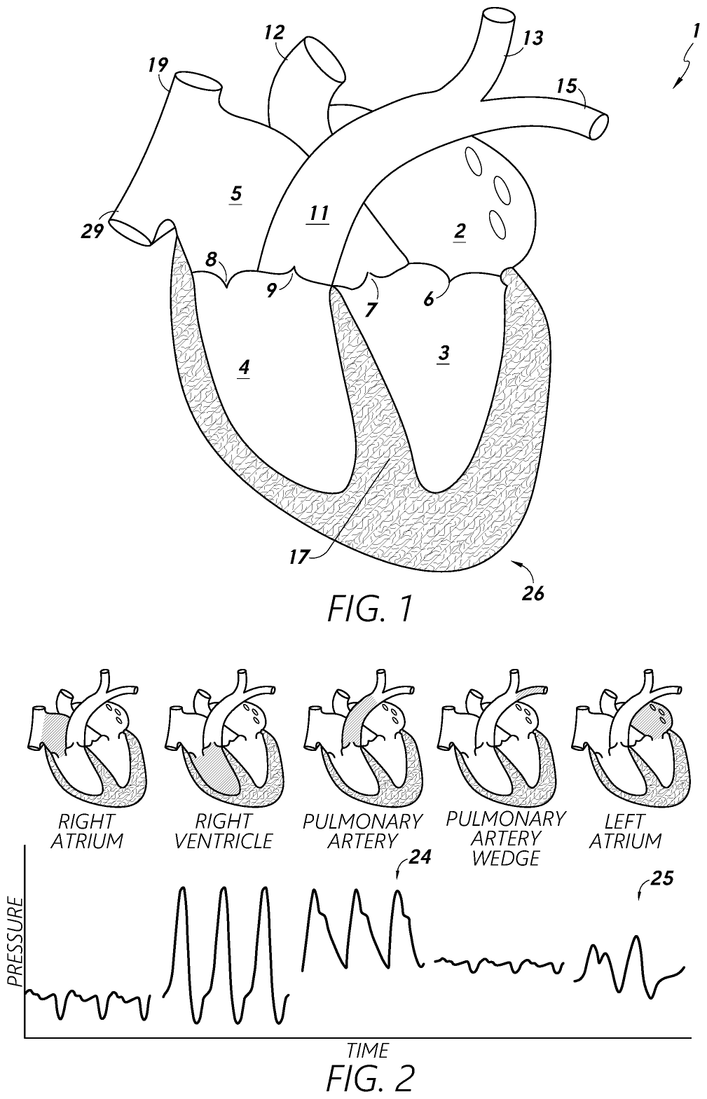

FIG. 1 illustrates an example representation of a heart 1 having various features relevant to certain embodiments of the present inventive disclosure. The heart 1 includes four chambers, namely the left atrium 2, the left ventricle 3, the right ventricle 4, and the right atrium 5. In terms of blood flow, blood generally flows from the right ventricle 4 into the pulmonary artery via the pulmonary valve 9, which separates the right ventricle 4 from the pulmonary artery 11 and is configured to open during systole so that blood may be pumped toward the lungs and close during diastole to prevent blood from leaking back into the heart from the pulmonary artery 11.

The pulmonary artery 11 carries deoxygenated blood from the right side of the heart to the lungs. The pulmonary artery 11 includes a pulmonary trunk and left 15 and right 13 pulmonary arteries that branch off of the pulmonary trunk, as shown. In addition to the pulmonary valve 9, the heart 1 includes three additional valves for aiding the circulation of blood therein, including the tricuspid valve 8, the aortic valve 7, and the mitral valve 6. The tricuspid valve 8 separates the right atrium 5 from the right ventricle 4. The tricuspid valve 8 generally has three cusps or leaflets and may generally close during ventricular contraction (i.e., systole) and open during ventricular expansion (i.e., diastole). The mitral valve 6 generally has two cusps/leaflets and separates the left atrium 2 from the left ventricle 3. The mitral valve 6 is configured to open during diastole so that blood in the left atrium 2 can flow into the left ventricle 3, and, when functioning properly, closes during systole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

The heart valves may generally comprise a relatively dense fibrous ring, referred to herein as the annulus, as well as a plurality of leaflets or cusps attached to the annulus. Generally, the size of the leaflets or cusps may be such that when the heart contracts the resulting increased blood pressure produced within the corresponding heart chamber forces the leaflets at least partially open to allow flow from the heart chamber. As the pressure in the heart chamber subsides, the pressure in the subsequent chamber or blood vessel may become dominant and press back against the leaflets. As a result, the leaflets/cusps come in apposition to each other, thereby dosing the flow passage. Disfunction of a heart valve and/or associated leaflets (e.g., pulmonary valve disfunction) can result in valve leakage and/or other health complications.

The atrioventricular (i.e., mitral and tricuspid) heart valves generally are coupled to a collection of chordae tendineae and papillary muscles (not shown) for securing the leaflets of the respective valves to promote and/or facilitate proper coaptation of the valve leaflets and prevent prolapse thereof. The papillary muscles, for example, may generally comprise finger-like projections from the ventricle wall. The valve leaflets are connected to the papillary muscles by the chordae tendineae. A wall of muscle 17, referred to as the septum, separates the left 2 and right 5 atria and the left 3 and right 4 ventricles.

Health Conditions Associated with Cardiac Pressure and Other Parameters

As referenced above, certain physiological conditions or parameters associated with the cardiac anatomy can impact the health of a patient. For example, congestive heart failure is a condition associated with the relatively slow movement of blood through the heart and/or body, which causes the fluid pressure in one or more chambers of the heart to increase. As a result, the heart does not pump sufficient oxygen to meet the body's needs. The various chambers of the heart may respond to pressure increases by stretching to hold more blood to pump through the body or by becoming relatively stiff and/or thickened. The walls of the heart can eventually weaken and become unable to pump as efficiently. In some cases, the kidneys may respond to cardiac inefficiency by causing the body to retain fluid. Fluid build-up in arms, legs, ankles, feet, lungs, and/or other organs can cause the body to become congested, which is referred to as congestive heart failure. Acute decompensated congestive heart failure is a leading cause of morbidity and mortality, and therefore treatment and/or prevention of congestive heart failure is a significant concern in medical care.

Various methods for identifying and/or treating congestive heart failure involve the observation of worsening congestive heart failure symptoms and/or changes in body weight. However, such signs may appear relatively late and/or be relatively unreliable. For example, daily bodyweight measurements may vary significantly (e.g., up to 9% or more) and may be unreliable in signaling heart-related complications. Furthermore, treatments guided by monitoring signs, symptoms, weight, and/or other biomarkers have not been shown to substantially improve clinical outcomes. Therefore, direct or indirect measurement/monitoring of pressure and/or other parameter(s) using implant devices can provide better outcomes than purely observation-based solutions. For example, without direct or indirect monitoring of cardiac pressure, it can be difficult to infer, determine, or predict the presence or occurrence of congestive heart failure or other pathologies. Treatments or approaches not involving direct or indirect pressure monitoring may involve measuring or observing other present physiological conditions of the patient, such as measuring body weight, thoracic impedance, right heart catheterization, or the like.

### Cardiac Pressure Monitoring

Cardiac pressure monitoring in accordance with embodiments of the present disclosure may provide a proactive intervention mechanism for preventing or treating congestive heart failure. Generally, increases in ventricular filling pressures associated with diastolic and/or systolic heart failure can occur prior to the occurrence of symptoms that lead to hospitalization. For example, cardiac pressure indicators may present weeks prior to hospitalization with respect to some patients. Therefore, pressure monitoring systems in accordance with embodiments of the present disclosure may advantageously be implemented to reduce instances of hospitalization by guiding the appropriate or desired titration and/or administration of medications before the onset of heart failure.

Dyspnea represents a cardiac pressure indicator characterized by shortness of breath or the feeling that one cannot breathe well enough. Dyspnea may result from elevated atrial pressure, which may cause fluid buildup in the lungs from pressure back-up. Pathological dyspnea can result from congestive heart failure. However, a significant amount of time may elapse between the time of initial pressure elevation and the onset of dyspnea, and therefore symptoms of dyspnea may not provide sufficiently-early signaling of elevated atrial pressure. By monitoring pressure directly according to embodiments of the present disclosure, normal ventricular filling pressures may advantageously be maintained, thereby preventing or reducing effects of heart failure, such as dyspnea.

As referenced above, with respect to cardiac pressures, pressure elevation in the left atrium may be particularly correlated with heart failure. FIG. 2 illustrates example pressure waveforms associated with various chambers and vessels of the heart according to one or more embodiments. The various waveforms illustrated in FIG. 2 may represent waveforms obtained using right heart catheterization to advance one or more pressure sensors to the respective illustrated and labeled chambers or vessels of the heart. Pressure sensor devices disclosed herein may be implanted in any of the chambers/vessels shown in FIG. 2 for obtaining pressure data related to the respective chamber/vessel. As illustrated in FIG. 2, the waveform 25, which represents left atrial pressure, may be considered to provide the best feedback for early detection of congestive heart failure. Furthermore, there may generally be a relatively strong correlation between increases in left atrial pressure and pulmonary congestion.

Left atrial pressure may generally correlate well with left ventricular end-diastolic pressure. However, although left atrial pressure and end-diastolic pulmonary artery pressure can have a significant correlation, such correlation may be weakened when the pulmonary vascular resistance becomes elevated. That is, pulmonary artery pressure generally fails to correlate adequately with left ventricular end-diastolic pressure in the presence of a variety of acute conditions, which may include certain patients with congestive heart failure. For example, pulmonary hypertension, which affects approximately 25% to 83% of patients with heart failure, can affect the reliability of pulmonary artery pressure measurement for estimating left-sided filling pressure. Therefore, pulmonary artery pressure measurement alone, as represented by the waveform 24, may be an insufficient or inaccurate indicator of left ventricular end-diastolic pressure, particularly for patients with co-morbidities, such as lung disease and/or thromboembolism. Left atrial pressure may further be correlated at least partially with the presence and/or degree of mitral regurgitation.

In some solutions, pulmonary capillary wedge pressure can be measured as a surrogate of left atrial pressure. For example, a pressure sensor may be disposed or implanted in the pulmonary artery, and readings associated therewith may be used as a surrogate for left atrial pressure. However, with respect to catheter-based pressure measurement in the pulmonary artery or certain other chambers or regions of the heart, use of invasive catheters may be required to maintain such pressure sensors, which may be uncomfortable or difficult to implement. Furthermore, certain lung-related conditions may affect pressure readings in the pulmonary artery, such that the correlation between pulmonary artery pressure and left atrial pressure may be undesirably attenuated. As an alternative to pulmonary artery pressure measurement, pressure measurements in the right ventricle outflow tract may relate to left atrial pressure as well, However, the correlation between such pressure readings and left atrial pressure may not be sufficiently strong to be utilized in congestive heart failure diagnostics, prevention, and/or treatment. The present disclosure provides systems, devices, and methods for packaging implantable pressure sensors configured to provide direct measurements of pressure conditions at the implantation site.

Additional solutions may be implemented for deriving or inferring left atrial pressure. For example, the E/A ratio, which is a marker of the function of the left ventricle of the heart representing the ratio of peak velocity blood flow from gravity in early diastole (the E wave) to peak velocity flow in late diastole caused by atrial contraction (the A wave), can be used as a surrogate for measuring left atrial pressure. The E/A ratio may be determined using echocardiography or other imaging technology; generally, abnormalities in the E/A ratio may suggest that the left ventricle cannot fill with blood properly in the period between contractions, which may lead to symptoms of heart failure, as explained above. However, E/A ratio determination generally does not provide absolute pressure measurement values.

Direct left atrial pressure readings may be relatively less likely to be distorted or affected by other conditions, such as respiratory conditions or the like, compared to the other pressure waveforms shown in FIG. 2. Generally, left atrial pressure may be significantly predictive of heart failure, such as up two weeks before manifestation of heart failure. For example, increases in left atrial pressure, and both diastolic and systolic heart failure, may occur weeks prior to hospitalization, and therefore knowledge of such increases may be used to predict the onset of congestive heart failure, such as acute debilitating symptoms of congestive heart failure.

Cardiac pressure monitoring, such as left atrial pressure monitoring, can provide a mechanism to guide administration of medication to treat and/or prevent congestive heart failure. Such treatments may advantageously reduce hospital readmissions and morbidity, as well as provide other benefits. An implanted pressure sensor in accordance with embodiments of the present disclosure may be used to predict heart failure up two weeks or more before the manifestation of symptoms or markers of heart failure (e.g., dyspnea). When heart failure predictors are recognized using cardiac pressure sensor embodiments in accordance with the present disclosure, certain prophylactic measures may be implemented, including medication intervention, such as modification to a patient's medication regimen, which may help prevent or reduce the effects of cardiac dysfunction. Direct pressure measurement in the left atrium can advantageously provide an accurate indicator of pressure buildup that may lead to heart failure or other complications. For example, trends of atrial pressure elevation may be analyzed or used to determine or predict the onset of cardiac dysfunction, wherein drug or other therapy may be augmented to cause reduction in pressure and prevent or reduce further complications.

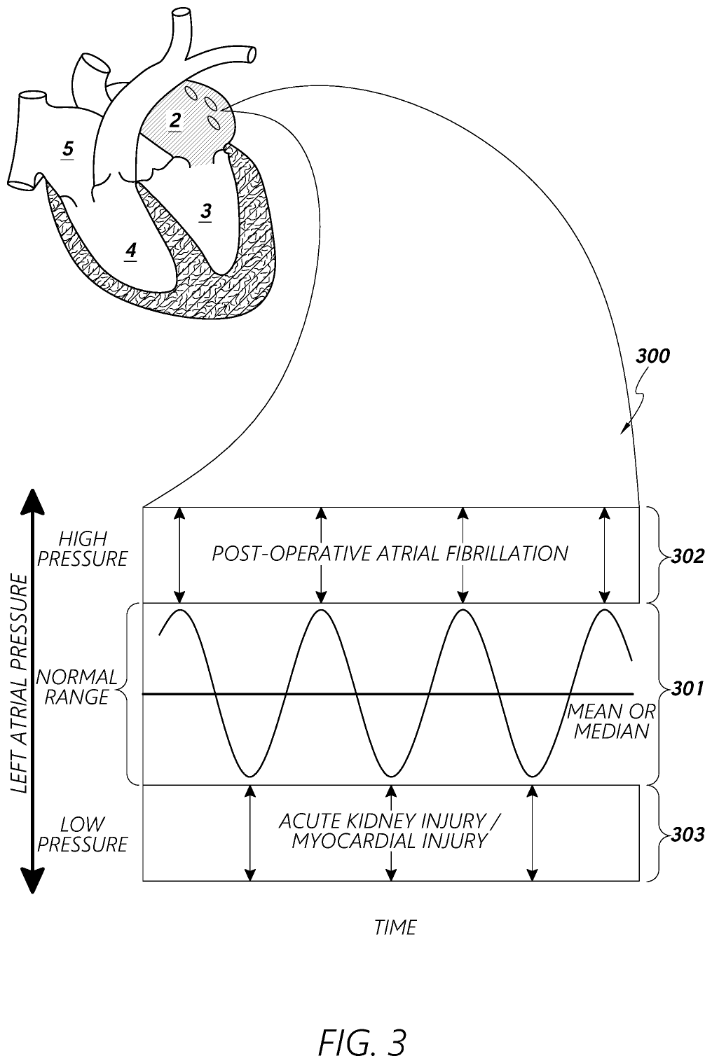

FIG. 3 illustrates a graph 300 showing left atrial pressure ranges including a normal range 301 of left atrial pressure that is not generally associated with substantial risk of postoperative atrial fibrillation, acute kidney injury, myocardial injury, heart failure and/or other health conditions. Systems, devices, and methods disclosed herein for monitoring cardiac pressure conditions using implantable pressure sensor devices can be implemented to determine whether a patient's left atrial pressure is within the normal range 301, above the normal range 303, or below the normal range 302. For detected left atrial pressure above the normal range, which may be correlated with an increased risk of heart failure, embodiments of the present disclosure as described in detail below can inform efforts to reduce the left atrial pressure until it is brought within the normal range 301. Furthermore, for detected left atrial pressure that is below the normal range 301, which may be correlated with increased risks of acute kidney injury, myocardial injury, and/or other health complications, embodiments of the present disclosure as described in detail below can serve to facilitate efforts to increase the left atrial pressure to bring the pressure level within the normal range 301.

### Implantable Pressure Sensor Devices

Pressure sensors that can be used in medical implant applications include sensors utilizing micro-electromechanical system (MEMS) technology. Such devices may combine relatively small mechanical and electrical components on a substrate, such as silicone or other semiconductor substrate, and may incorporate deformable membranes that are used to measure pressure-induced deflection thereof, wherein the degree of deflection of the membrane is indicative of pressure conditions to which the sensor membrane is exposed at the implant location.

MEMS sensors may be desirable for cardiac implant applications due to their relatively small form factors and packaging. For example, MEMS pressure sensor devices may be considered relatively small, stable, and cost-effective devices, wherein such characteristics can accommodate the relatively constrained space and/or cost requirements of certain implant devices. MEMS pressure sensor devices in accordance with embodiments of the present disclosure can be fabricated in silicon using certain doping and/or etching processes. Such processes may be performed at a chip-scale, providing relatively small devices that can be co-packaged with certain signal-conditioning electronics, including passive and/or active devices. For example, electronic circuitry electrically coupled to a MEMS pressure sensor in connection with any of the embodiments disclosed herein may comprise signal amplification, analog-to-digital conversion, filtering, and/or other signal processing functionality and control circuitry. The term “control circuitry” is used herein according to its broad and ordinary meaning, and may refer to any collection of processors, processing circuitry, processing modules/units, chips, dies (e.g., semiconductor dies including come or more active and/or passive devices and/or connectivity circuitry), microprocessors, micro-controllers, digital signal processors, microcomputers, central processing units, field programmable gate arrays, programmable logic devices, state machines (e.g., hardware state machines), logic circuitry, analog circuitry, digital circuitry, and/or any device that manipulates signals (analog and/or digital) based on hard coding of the circuitry and/or operational instructions. Control circuitry referenced herein may further comprise one or more, storage devices, which may be embodied in a single memory device, a plurality of memory devices, and/or embedded circuitry of a device. Such data storage may comprise read-only memory, random access memory, volatile memory, non-volatile memory, static memory, dynamic memory, flash memory, cache memory, data storage registers, and/or any device that stores digital information. It should be noted that in embodiments in which control circuitry comprises a hardware and/or software state machine, analog circuitry, digital circuitry, and/or logic circuitry, data storage device(s)/register(s) storing any associated operational instructions may be embedded within, or external to, the circuitry comprising the state machine, analog circuitry, digital circuitry, and/or logic circuitry.

Various types of pressure sensors can be built using MEMS technology, including piezoresistive pressure sensors and capacitive pressure sensors. Such sensors generally include an at least partially flexible layer that serves as a deformable membrane that is configured to act as a diaphragm that deflects under pressure. Piezoresistive and capacitive sensors use different mechanisms to measure the displacement of such diaphragm components.

With respect to piezoresistive MEMS pressure sensors, certain conductive sensing elements may be fabricated directly onto a diaphragm of the device, wherein changes in the electrical resistance of such conductor(s) can be determined to indicate a measure of pressure applied to the diaphragm. Generally, the change in resistance may be proportional to the strain on the conductor(s), wherein the change in resistance of the conductor(s) is related to the change in length of the conductor(s) induced by deflection of the diaphragm on which the conductor(s) are disposed.

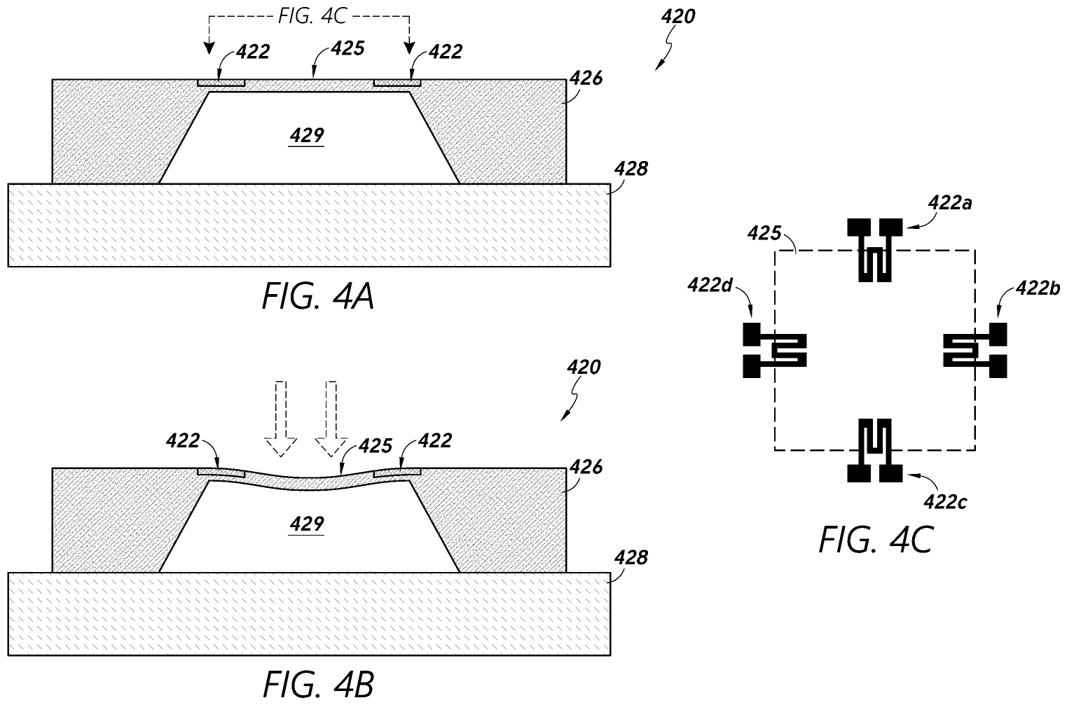

FIG. 4A shows a side view of a piezoresistive MEMS sensor device 420 in accordance with one or more embodiments of the present disclosure. FIG. 4B shows a view of the piezoresistive sensor device 420, wherein the diaphragm 425 of the device 420 is in a deflected configuration caused by pressure conditions to which the diaphragm 425 is exposed. The diaphragm 425 may be formed from a substrate material 426, such as silicon or other semiconductor or material. For example, a trench or cavity 429 may be etched or formed in the substrate 426 to produce a relatively thin membrane for the diaphragm 425.

The diaphragm 425 may have one or more conductive traces or elements 422 disposed thereon and/or applied thereto. For example, the conductive elements 422 may comprise traces of metal or other electrical conductor, wherein one or more length portions of the conductor(s) extend over the diaphragm 425, such that deflection of the diaphragm 425 causes one or more portions of the conductor(s) 422 to elongate/lengthen, thereby altering the electrical resistance/impedance thereof When the diaphragm 425 deflects, as shown in FIG. 4B, electrical current and/or voltage through the conductive element(s) 422 may be measured to determine respective resistances/impedances thereof, thereby providing a measurement indicating a degree of deflection of the diaphragm 425; such deflection indicates the environmental pressure experienced by the diaphragm 425. The diaphragm 425 may comprise any material(s), including but not limited to metal, ceramic, silicon, and the like.

FIG. 4C shows a schematic view of the diaphragm 425 showing an example arrangement of the conductive element(s) 422. Although four conductive elements/traces are shown in FIG. 4C, it should be understood that embodiments of piezoresistive pressure sensor devices may include any number or arrangement of diaphragm conductive elements. The configuration of the conductors 422 shown in FIG. 4C represents an example of a bridge-type pressure sensor. Generally, the linearity of the sensor device 420 may depend at least in part on the stability of the diaphragm 425 over the relevant measurement range and/or the linearity of the conductive elements 422.

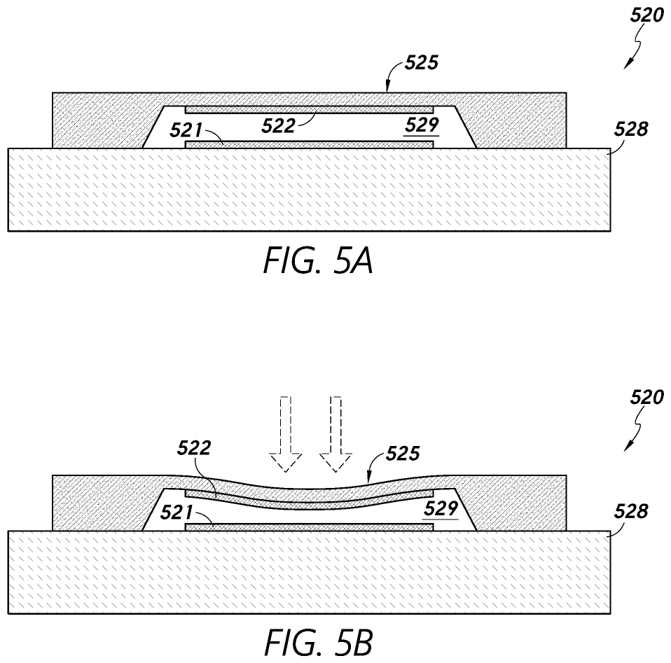

FIGS. 5A and SB show side views of a capacitive MEMS pressure sensor device 520 with a straight and deflected diaphragm component 525, respectively, in accordance with one or more embodiments of the present disclosure. For capacitive MEMS pressure sensors, one or more conductive layers 521, 522 may be deposited/applied on/to the diaphragm 525 and at, the bottom of a cavity 529 behind/below the diaphragm 525, respectively, to create a capacitor. For example; in some implementations, the diaphragm itself 525 may comprise conductive material serving as a capacitor electrode, or a separate conductive electrode may be applied to a side of the diaphragm 525 that is exposed within the cavity 529. That is, the sensor device 520 may comprise one rigid plate electrode 522 and one flexible membrane electrode 525. With the area of such electrodes being fixed, the capacitance between the electrodes may be proportional to the distance(s) between them.

As shown in FIG. 5B, inward/downward deflection/deformation of the diaphragm 525 may change the spacing between the conductors 521, 522 over at least a portion of the diaphragm 525, thereby changing the capacitance of the capacitor formed between the diaphragm 525 and the base electrode 522. Such change in capacitance may be measured by coupling the sensor device 520 to a tuned circuit, for example, which may have a fundamental frequency that is proportional to the degree of deflection of the diaphragm 525. The diaphragm 525 may comprise any material(s), including but not limited to metal, ceramic, silicon, and the like.

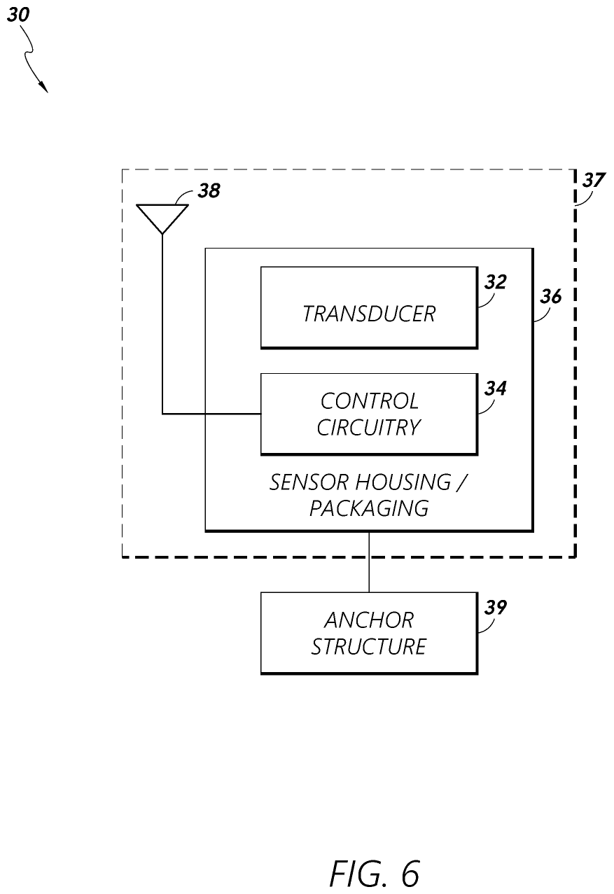

In some implementations, the present disclosure relates to sensors associated or integrated with anchoring implant structures, which may include shunt structures or other implant structures mechanically coupled to the sensor device. FIG. 6 is a block diagram illustrating an implant device 30 comprising a sensor device 37 coupled to certain anchoring structure 39, which may be configured to anchor in and/or to one or more biological tissue walls. The sensor device 37 may be, for example, a pressure sensor according to any of the embodiments disclosed herein. In some embodiments, the sensor 37 comprises a transducer 32, such as a MEMS pressure transducer, as well as certain control circuitry 34, which may be embodied in, for example, an application-specific integrated circuit (ASIC) and/or one or more passive devices (e.g., resistors, capacitors, inductors, etc.).

The control circuitry 34 may be configured to process signals received from the transducer 32, and/or communicate signals associated therewith wirelessly through biological tissue using the antenna 38. The antenna 38 may comprise one or more coils or loops of conductive material, such as copper wire or the like, or piezoelectric resonator(s), or other wireless signal transmission component(s). In some embodiments, at least a portion of the transducer 32, control circuitry 34, and/or the antenna 38 are at least partially disposed or contained within certain sensor housing/packaging 36 structure, which may comprise any type of material and may advantageously be at least partially hermetically sealed. For example, the housing/packaging 36 may comprise one or more tubes, cans, substrates/boards or other structures comprising glass or other rigid material(s) in some embodiments, which may provide mechanical stability and/or protection for the components housed therein. In some embodiments, the housing/packaging 36 is at least partially flexible, For example, the housing/packaging may comprise polymer or other flexible structure/material, which may advantageously allow for folding, bending, or collapsing of aspects of the sensor 37 to allow for passage thereof through a catheter or other introducing means.

The transducer 32 may comprise any type of sensor means or mechanism. For example, the transducer 32 may be a force-collector-type pressure sensor. In some embodiments, the transducer 32 comprises a diaphragm, piston, Bourdon tube, bellows, or other strain- or deflection-measuring component(s) to measure strain or deflection applied over an area/surface thereof The transducer 32 may be associated with the housing/packing 36, such that at least a portion thereof is contained within or attached to the housing/packaging 36. In some embodiments, the transducer 32 comprises or is a component of a piezoresistive MEMS pressure sensor, which may be configured to use bonded or formed conductors to detect strain due to applied pressure, wherein resistance increases as pressure deforms the component/material, as described above in connection with FIGS. 4A-4C. Alternatively, the transducer may comprise or be a component of a capacitive pressure sensor, as described above in connection with FIGS. 5A and 5B. The transducer 32 may incorporate any type of material, including but not limited to silicon (e.g., monocrystalline), polysilicon thin film, bonded metal foil, thick film, silicon-on-sapphire, sputtered thin film, and/or the like.

In some embodiments, the transducer 32 comprises or is a component of an electromagnetic pressure sensor, which may be configured to measure the displacement of a diaphragm by means of changes in inductance, linear variable displacement transducer (LVDT) functionality, Hall Effect, or eddy current sensing. In some embodiments, the transducer 32 comprises or is a component of a piezoelectric strain sensor. For example, such a sensor may determine strain (e.g., pressure) on a sensing mechanism based on the piezoelectric effect in certain materials, such as quartz.

In some embodiments, the transducer 32 comprises or is a component of a strain gauge. For example, a strain gauge embodiment may comprise a pressure sensitive element on or associated with an exposed surface of the transducer 32. In some embodiments, a metal strain gauge is adhered to a surface of the sensor, or a thin-film gauge may be applied on the sensor by sputtering or other technique. The measuring element or mechanism may comprise a diaphragm or metal foil. The transducer 32 may comprise any other type of sensor or pressure sensor, such as optical, potentiometric, resonant, thermal, ionization, or other types of strain or pressure sensors.

The transducer 32 can comprise one or more MEMS pressure sensor devices, as described in detail herein, mounted in or to a can-type package, board, and/or the like. Furthermore, the transducer 32 may be covered in one or more layers of transduction medium and/or biocompatibility material, as described in detail below. In some embodiments, the transducer(s) 32 is/are electrically and/or communicatively coupled to the control circuitry 34, which may comprise one or more application-specific integrated circuit (ASIC) microcontrollers or chips. The control circuitry 34 can further include one or more discrete electronic components, such as tuning capacitors, resistors, diodes, inductors, or the like.

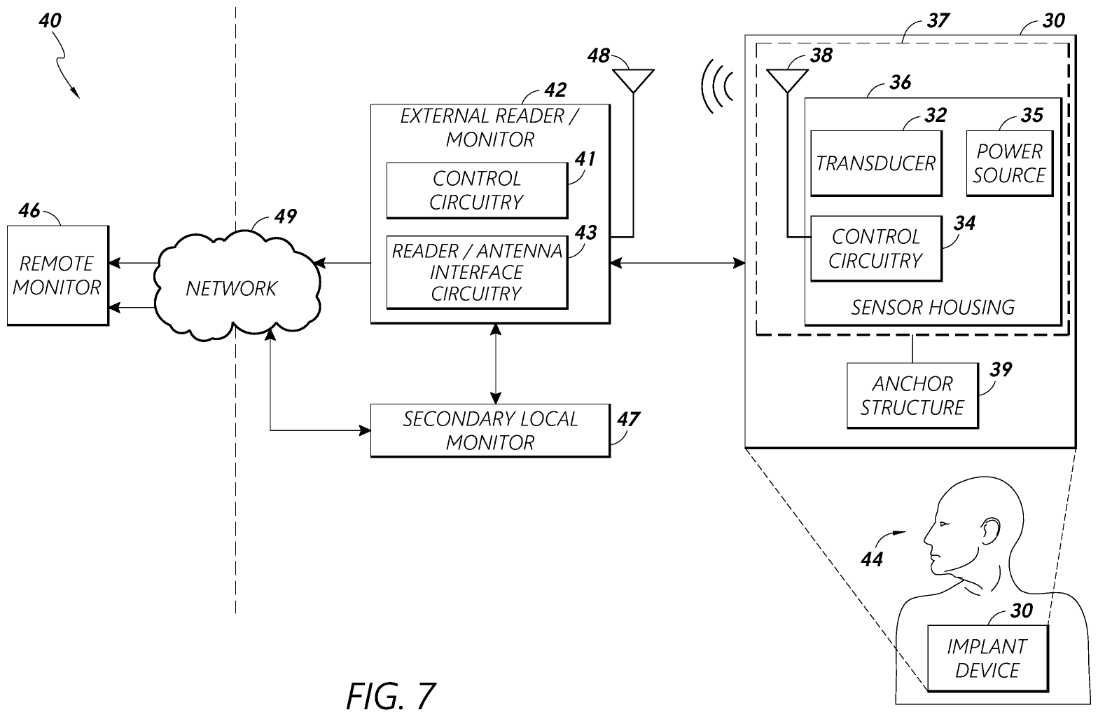

FIG. 7 shows a system 40 for monitoring one or more physiological parameters (e.g., left atrial pressure and/or volume) in a patient 44 according to one or more embodiments. The patient 44 can have a medical implant device 30 implanted in, for example, the heart (not shown), or associated physiology, of the patient 44. For example, the implant device 30 can be implanted at least partially within the left atrium of the patient's heart. The implant device 30 can include one or more sensor transducers 32, such as one or more microelectromechanical system (MEMS) devices, such as MEMS pressure sensors, or other type of sensor transducer.

In certain embodiments, the monitoring system 40 can comprise at least two subsystems, including an implantable internal subsystem or device 30 that includes the sensor transducer(s) 32, as well as control circuitry 34 comprising one or more microcontroller(s), discrete electronic component(s), and one or more power and/or data transmitters) 38 (e.g., antenna coils). The monitoring system 40 can further include an external (e.g., non-implantable) subsystem that includes an external reader 42 (e.g., coil), which may include a wireless transceiver that is electrically and/or communicatively coupled to certain control circuitry. In certain embodiments, both the internal and external subsystems include a corresponding coil antenna for wireless communication and/or power delivery through patient tissue disposed therebetween. The sensor implant device 30 can be any type of implant device. For example, in some embodiments, the implant device 30 comprises a pressure sensor integrated with another functional implant structure, such as a prosthetic shunt or stent device/structure.

Certain details of the implant device 30 are illustrated in the enlarged block 30 shown. The implant device 30 can comprise certain anchoring structure as described herein. For example, the anchor structure 39 can include a percutaneously deliverable shunt device configured to be secured to and/or in a tissue wall (e.g., interatrial septum, coronary sinus) to provide a flow path between two chambers and/or vessels of the heart, as described in greater detail throughout the present disclosure. Although certain components are illustrated in FIG. 7 as part of the implant device 30, it should be understood that the sensor implant device 30 may only comprise a subset of the illustrated components/modules and can comprise additional components/modules not illustrated. The implant device 30 may represent an embodiment of the implant device shown in FIG. 4, and vice versa. The implant device 30 can advantageously include one or more sensor transducers 32, which can be configured to provide a response indicative of one or more physiological parameters of the patient 44, such as atrial pressure. Although pressure transducers are described, the sensor transducer(s) 32 can comprise any suitable or desirable types of sensor transducer(s) for providing signals relating to physiological parameters or conditions associated with the implant device 30 and/or patient 44.

The transducer 32 can comprise one or more MEMS pressure sensor devices, as described in detail herein, mounted in or to a can-type package, board, and/or the like. Furthermore, the transducer 32 may be covered in one or more layers of transduction medium and/or biocompatibility material, as described in detail below. In some embodiments, the transducer(s) 32 is/are electrically and/or communicatively coupled to the control circuitry 34, which may comprise one or more application-specific integrated circuit (ASIC) microcontrollers or chips. The control circuitry 34 can further include one or more discrete electronic components, such as tuning capacitors, resistors, diodes, inductors, or the like.

In certain embodiments, the sensor transducer(s) 32 can be configured to generate electrical signals that can be wirelessly transmitted to a device outside the patient's body, such as the illustrated local external monitor system 42. In order to perform such wireless data transmission, the implant device 30 can include radio frequency (RF) (or other frequency band) transmission circuitry, such as signal processing circuitry and one or more antennas 38. The antenna 38 can comprise an internal antenna coil implanted within the patient. The control circuitry 34 may comprise any type of transceiver circuitry configured to transmit an electromagnetic signal, wherein the signal can be radiated by the antenna 38, which may comprise one or more conductive wires, coils, plates, or the like. The control circuitry 34 of the implant device 30 can comprise, for example, one or more chips or dies configured to perform some amount of processing on signals generated and/or transmitted using the device 30. However, due to size, cost, and/or other constraints, the implant device 30 may not include independent processing capability in some embodiments.

The wireless signals generated by the implant device 30 can be received by the local external monitor device or subsystem 42, which can include a reader/antenna-interface circuitry module 43 configured to receive the wireless signal transmissions from the implant device 30, which is disposed at least partially within the patient 44. For example, the module 43 may include transceiver device(s)/circuitry.

The external local monitor 42 can receive the wireless signal transmissions and/or provide wireless power using an external antenna 48, such as a wand device. The reader/antenna-interface circuitry 43 can include radio-frequency (RF) (or other frequency band) front-end circuitry configured to receive and amplify the signals from the implant device 30, wherein such circuitry can include one or more filters (e.g., band-pass filters), amplifiers (e.g., low-noise amplifiers), analog-to-digital converters (ADC) and/or digital control interface circuitry, phase-locked loop (PLL) circuitry, signal mixers, or the like. The reader/antenna-interface circuitry 43 can further be configured to transmit signals over a network 49 to a remote monitor subsystem or device 46. The RF circuitry of the reader/antenna-interface circuitry 43 can further include one or more of digital-to-analog converter (DAC) circuitry, power amplifiers, low-pass filters, antenna switch modules, antennas or the like for treatment/processing of transmitted signals over the network 49 and/or for receiving signals from the implant device 30. In certain embodiments, the local monitor 42 includes control circuitry 41 for performing processing of the signals received from the implant device 30. The local monitor 42 can be configured to communicate with the network 49 according to a known network protocol, such as Ethernet, Wi-Fi, or the like. In certain embodiments, the local monitor 42 comprises a smartphone, laptop computer, or other mobile computing device, or any other type of computing device.

In certain embodiments, the implant device 30 includes some amount of volatile and/or non-volatile data storage. For example, such data storage can comprise solid-state memory utilizing an array of floating-gate transistors, or the like. The control circuitry 34 may utilize data storage for storing sensed data collected over a period of time, wherein the stored data can be transmitted periodically to the local monitor 42 or another external subsystem. In certain embodiments, the implant device 30 does not include any data storage. The control circuitry 34 may be configured to facilitate wireless transmission of data generated by the sensor transducer(s) 32, or other data associated therewith. The control circuitry 34 may further be configured to receive input from one or more external subsystems, such as from the local monitor 42, or from a remote monitor 46 over, for example, the network 49. For example, the implant device 30 may be configured to receive signals that at least partially control the operation of the implant device 30, such as by activating/deactivating one or more components or sensors, or otherwise affecting operation or performance of the implant device 30.

The one or more components of the implant device 30 can be powered by one or more power sources 35. Due to size, cost and/or electrical complexity concerns, it may be desirable for the power source 35 to be relatively minimalistic in nature. For example, high-power driving voltages and/or currents in the implant device 30 may adversely affect or interfere with operation of the heart or other body part associated with the implant device. In certain embodiments, the power source 35 is at least partially passive in nature, such that power can be received from an external source wirelessly by passive circuitry of the implant device 30, such as through the use of short-range, or near-field wireless power transmission, or other electromagnetic coupling mechanism, For example, the local monitor 42 may serve as an initiator that actively generates an RF field that can provide power to the implant device 30, thereby allowing the power circuitry of the implant device to take a relatively simple form factor. In certain embodiments, the power source 35 can be configured to harvest energy from environmental sources, such as fluid flow, motion, or the like. Additionally or alternatively, the power source 35 can comprise a battery, which can advantageously be configured to provide enough power as needed over the monitoring period (e.g., 3, 5, 10, 20, 30, 40, or 90 days, or other period of time).

In some embodiments, the local monitor device 42 can serve as an intermediate communication device between the implant device 30 and the remote monitor 46. The local monitor device 42 can be a dedicated external unit designed to communicate with the implant device 30. For example, the local monitor device 42 can be a wearable communication device, or other device that can be readily disposed in proximity to the patient 44 and implant device 30. The local monitor device 42 can be configured to continuously, periodically, or sporadically interrogate the implant device 30 in order to extract or request sensor-based information therefrom. In certain embodiments, the local monitor 42 comprises a user interface, wherein a user can utilize the interface to view sensor data, request sensor data, or otherwise interact with the local monitor system 42 and/or implant device 30.

The system 40 can include a secondary local monitor 47, which can be, for example, a desktop computer or other computing device configured to provide a monitoring station or interface for viewing and/or interacting with the monitored cardiac pressure data. In an embodiment, the local monitor 42 can be a wearable device or other device or system configured to be disposed in close physical proximity to the patient and/or implant device 30, wherein the local monitor 42 is primarily designed to receive/transmit signals to and/or from the implant device 30 and provide such signals to the secondary local monitor 47 for viewing, processing, and/or manipulation thereof. The external local monitor system 42 can be configured to receive and/or process certain metadata from or associated with the implant device 30, such as device ID or the like, which can also be provided over the data coupling from the implant device 30.

The remote monitor subsystem 46 can be any type of computing device or collection of computing devices configured to receive, process and/or present monitor data received over the network 49 from the local monitor device 42, secondary local monitor 47, and/or implant device 30. For example, the remote monitor subsystem 46 can advantageously be operated and/or controlled by a healthcare entity, such as a hospital, doctor, or other care entity associated with the patient 44. Although certain embodiments disclosed herein describe communication with the remote monitor subsystem 46 from the implant device indirectly through the local monitor device 42, in certain embodiments, the implant device 30 can comprise a transmitter capable of communicating over the network 49 with the remote monitor subsystem 46 without the necessity of relaying information through the local monitor device 42.

In certain embodiments, the antenna 48 of the external monitor system 42 comprises an external coil antenna that is matched and/or tuned to be inductively paired with the antenna 38 of the internal implant 30. In some embodiments, the implant device 30 is configured to receive wireless ultrasound power charging and/or data communication between from the external monitor system 42. As referenced above, the local external monitor 42 can comprise a wand or other hand-held reader. In some embodiments, the antenna 48 comprises a piezoelectric crystal.

### Implantable Pressure Sensor Packaging

Sensor implant devices in accordance with aspects of the present disclosure can include various functional components and/or assemblies. For example, such implants may generally include a sensing element, various embedded electronics, which may comprise one or more application-specific integrated circuit chips (ASIC), and/or certain communication and/or energy-receiving/providing components.

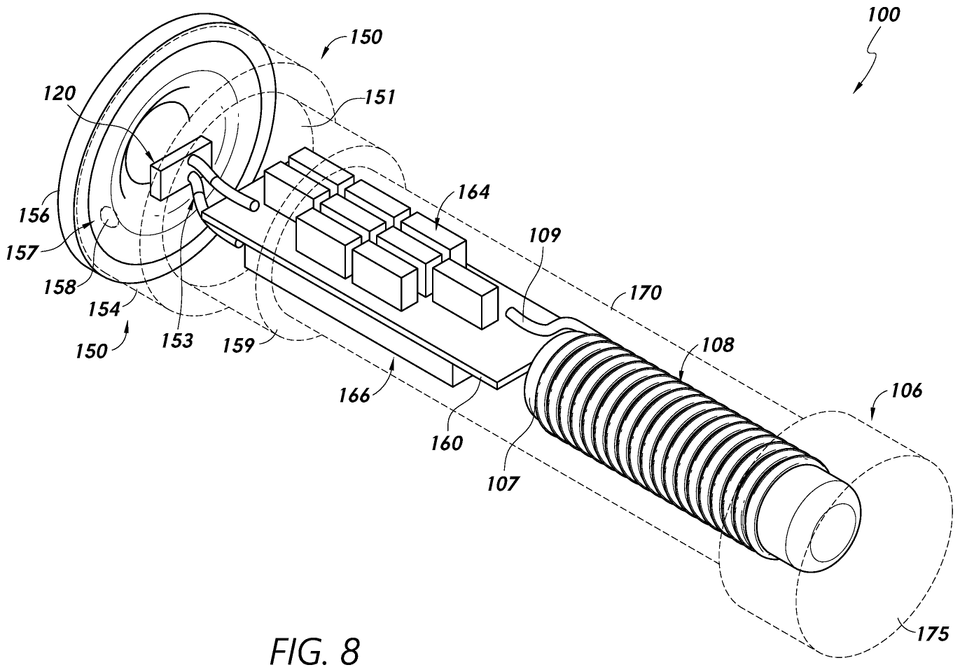

FIG. 8 is a perspective view of a sensor implant device 100 in accordance with one or more embodiments of the present disclosure. The sensor implant device 100 includes a wireless telemetry component 108. The wireless telemetry functionality associated with implant devices disclosed herein can be configured to transmit and/or receive radiofrequency electromagnetic signals, ultrasound signals, and/or other wireless signal type. Although wireless data and/or energy transmission is described in connection with various embodiments disclosed herein, it should be understood that such embodiments may be implemented using wired data and/or power transmission features. For example, the sensor implant devices disclosed herein may be implemented as components of a catheter assembly, wherein such devices may not be intended for long-term implantation, but rather may be positioned in target anatomy through advancements and positioning of such catheter and/or distal end thereof.

The implant device 100 includes a pressure sensor support can/cup 150, which may have a MEMS pressure sensor device 120 mounted or secured therein. The implant device 100 further includes a circuit board 160 (e.g., printed circuit board) having certain electronics mounted thereto, including one or more passive devices 164 and/or application-specific integrated circuits (ASIC) 166, which may be on either or both sides of the circuit board 160.

In some embodiments, a wire coil antenna 108 or other type of transmitter/receiver is electrically coupled to the circuit board 160 and/or electrical components mounted thereto. For example, in the illustrated embodiment, a conductive wire coil (e.g., copper wire) may be wound around a ferrite core 107 that is collinear or coplanar with the axis of the cylindrical sensor housing 170 in which it is at least partially disposed. The ferrite core 107, which may be referred to as a ferrite bead, block, choke, or electromagnetic interference (EMI) filter, may be configured to suppress relatively high-frequency electronic noise. For example, the ferrite core 107 may comprise iron, ceramic, or the like, and may employ relatively high-frequency current dissipation to prevent electromagnetic interference in one or more dimensions.

The various control circuitry components, including the printed circuit board 160 and antenna 108, may be maintained at least partially within a rigid housing 170, which is shown as a transparency in FIG. 8 for clarity. The tubular housing 170 may comprise ceramic, zirconia, glass, or other at least partially rigid structure that is hard enough to protect the internal components from damage during implantation and/or over prolonged exposure at the implantation site. The housing 170 may advantageously provide a moisture barrier to prevent moisture from penetrating the housing 170 and interacting with the components housed therein. The electronics housing 170 may further comprise material that is sufficiently transparent to radiofrequency electromagnetic radiation, which may be transmitted to and/or from the antenna 108 to allow for data and/or power communication with the implant device 100. In some embodiments, the sensor implant device 100 is configured to communicate data and/or power/energy through transmission of ultrasound signals and/or other some signal communication. Therefore, it may be desirable in some embodiments to construct the housing 170 from material that is sufficiently transparent to ultrasound and/or other some signals. In some embodiments, a back end 106 of the tube may be covered with a metal or other sealing component configured to seal the back opening of the tube housing 170.

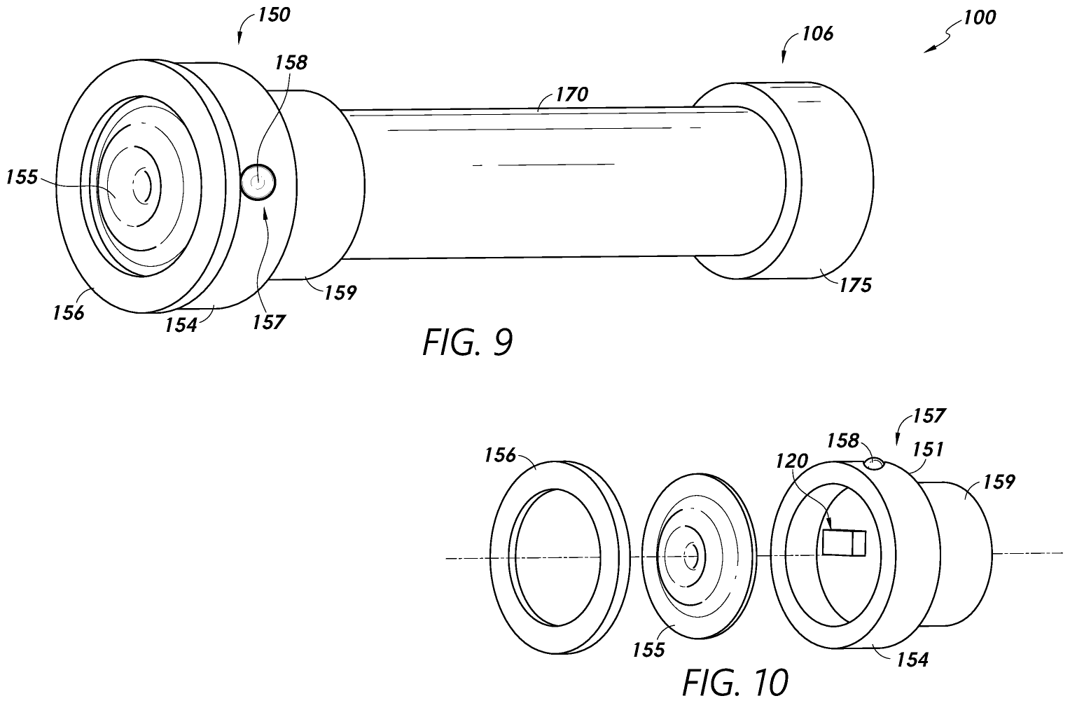

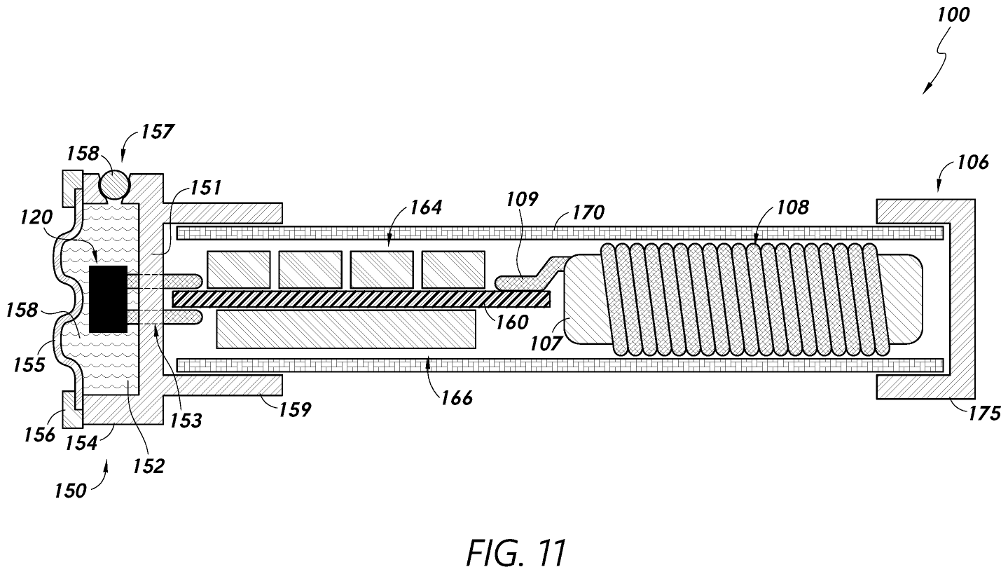

FIG. 9 shows a front and side perspective view of the sensor implant device 100 shown in FIG. 8, wherein a pressure membrane 155 on the front/distal end of the device is shown, which may be secured to the sensor can/cup 150 in some manner. Deflection of the membrane 155 may be translated to a diaphragm/membrane of a MEMS sensor device 120 disposed within the sensor can package 150 through a transduction medium, such as silicone oil. In some embodiments, the sensor can package 150 includes one or more side walls 154, and a cover component 156, which is configured to secure and seal the membrane 155 to the can package 150 in a fluid-tight configuration. FIG. 10 provides an exploded view of the sensor can package 150 of FIGS. 8 and 9 in accordance with one or more embodiments.

FIG. 11 shows a cross-sectional view of the sensor implant device 100 shown in FIGS. 8-10 in accordance with one or more embodiments. The view of FIG. 11 shows the membrane 155, which may comprise a corrugated metal disc or sheet, sealed to the can structure 150 to enclose a cavity or chamber 152 within the can package 150. In some embodiments, the chamber 152 is filled with a liquid material, such as silicone oil or the like, wherein such liquid is not compressible, such that inward deflection of the membrane 155 increases pressure within the chamber 152. In some embodiments, the membrane 155 comprises a corrugated metal foil cover, although other materials are possible. The corrugated topology of the membrane 155 may facilitate deflection of the membrane 155 in a manner as to translate pressure to the sensor device 120.

Where the chamber 152 is filled with liquid, it may be desirable to fill the chamber such that no air or gas bubbles exist within the chamber 152. For example, air/gas may generally be pressure-compressible, such that the presence of air/gas within the chamber 152 may decrease the translation of pressure from deflection of the membrane 155 into pressure within the chamber 152. In some implementations, the chamber 152 may be filled through an inlet/port 157, which may be associated with a sidewall portion 154. For example, the inlet 27 may be used to pipe the liquid into the chamber 152, wherein the inlet 157 is sealed in some manner to prevent leakage of the fluid out of the chamber 152 and/or prevent gas or other matter from entering the chamber 152 after it has been filled. In some embodiments, a ball bearing 158 or other feature may be used to seal-off the inlet 157. In some implementations, the chamber 152 may be filled under vacuum conditions.

Various components of the sensor implant device 100, including the can package 150 and the proximal endcap 175, may be welded to the tubular housing 170. The base 151 of the can package 150 can include one or more through-holes 153, which may advantageously be fluid-sealed when the chamber 152 is filled with oil or other material.

As referenced, the membrane 155 may advantageously be hermetically sealed to the can package 150, wherein the silicone oil pressure transduction chamber 152 is sealed-off using one or more outer seals/covers 156. The sensor implant device 100 may be designed to meet certain packaging requirements for, for example, cardiac implantation or implantation within another target location of the body. Therefore, the configuration of the sensor implant device 100 may necessarily or advantageously protect the sensor 120 and/or circuitry components within the housing 170 from the environment of the implantation site (e.g., blood exposure environment).

The sensor implant device 100 may further be configured with certain biocompatibility features (e.g., coatings, coverings, treatments, etc.) to prevent tissue encapsulation of the sensor implant device 100 and/or membrane 15, which may result in loss of sensitivity. The sensor implant device 100 may further be configured to provide transduction of environmental pressure across the membrane 155 through the medium disposed within the chamber 152 to the sensor 120, electrically isolate electrical contact of the implant device 100, and/or protect the sensor device 120 and/or electrical connections and/or circuitry associated therewith from physical damage.

In accordance with some implementations, packaging of a sensor device in a manner as shown in FIGS. 8-11 may involve undesirably complicated sealing processes requiring manipulation of the sensor 120, hermetic sealing/welding, filling of the cavity/chamber 152 with pressure-transducing medium, and/or other complications. Certain embodiments of the present disclosure advantageously provide for pressure sensor encapsulation that requires less processing than solutions requiring welding of a metal membrane to a can package.

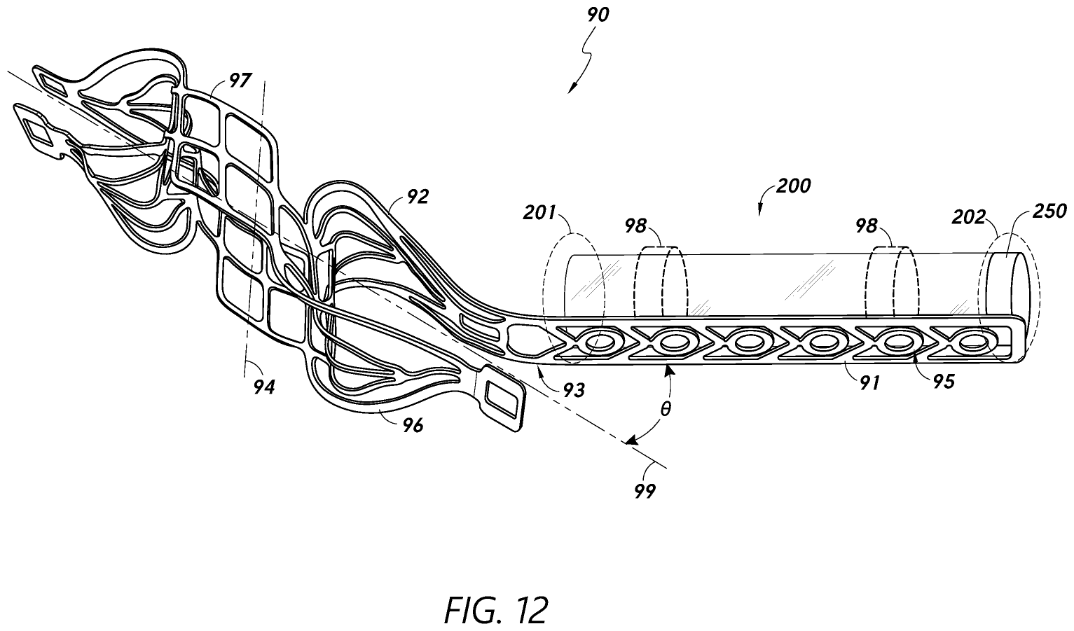

FIG. 12 illustrates a sensor implant device 90 having an integrated sensor 200 that is mechanically attached or fastened to a portion of a shunt/anchor structure 97. The shunt/anchor structure 97 comprises a sensor-support structure/arm 91, which may be a unitary form with the shunt/anchor structure 97. In some embodiments, the support 91 is an extension of, or otherwise associated with, an arm member 92 of the shunt/anchor structure 97. The sensor 100 may be attached to the support structure/arm 91 by any suitable or desirable attachment means, including adhesive attachment, or mechanical engagement. For example, the sensor support 91 may comprise or be associated with one or more retention features 98, which may comprise one or more clamps, straps, ties, sutures, collars, clips, tabs, or the like. Such retention features 98 may circumferentially encase or retain the sensor 100, or a portion thereof. In some embodiments, the sensor 100 may be attached to the sensor support 91 through the application of mechanical force, either through sliding the sensor 100 through the retention features 98 or through clipping, locking, or otherwise engaging the sensor 100 with the sensor support 91 by pressing or applying other mechanical force thereto. In some embodiments, the retention feature(s) 98 comprise one or more tabs that may be configured to pop-up or extend on one or more sides of the sensor support 91 for mechanical fastening. Such tabs may comprise memory metal (e.g., Nitinol) or other at least partially rigid material. In some embodiments, the sensor support 91 is at least partially non-rigid. For example, the sensor support 91 may comprise a non-rigid tether configured to float the sensor 100. Such configurations may advantageously allow for the sensor 100 to move with ambient blood flow.

In some embodiments, the sensor 100 is pre-attached to the sensor support 91 and/or integrated therewith prior to implantation. For example, in some embodiments, the sensor support 91 forms at least a portion of the housing of the sensor 100, such that the sensor support 91 and at least a portion of the housing of the sensor 100 are a unitary form.

In some embodiments, the angle or position of the sensor support 91 and/or sensor 100 relative to a longitudinal axis 99 of the shunt/anchor structure 97 is such that the sensor projects away from the longitudinal axis 99. For example, where the shunt/anchor structure 97 is engaged with biological tissue along the dimension of the longitudinal axis 99, the sensor 100 may advantageously project at least partially away from the biological tissue, such as into a chamber cavity (e.g., atrium of a heart). In some embodiments, the sensor support 91 is configured, or can be configured, substantially at a right angle or 90° orientation with respect to the axis 99, such that the sensor is substantially orthogonal to the longitudinal axis of the shunt. Such configurations may advantageously allow for the sensor element to be positioned a desirable distance away from shunted blood flow flowing through the flow path axis 94.

The sensor element 250 of the sensor 200 may be disposed or positioned at any location of the sensor 200. For example, the sensor element 250 may advantageously be disposed at or near a distal portion 250 of the sensor 200. Alternatively or additionally, a sensor element may be disposed or positioned at or near a proximal portion 201 of the sensor 100. The sensor element 250 may be packaged in accordance with various embodiments disclosed herein, including one or more transduction and/or biocompatibility layers, as described in detail below.

As demonstrated above with respect to the silicone-oil-filled can package of the sensor implant device shown in FIGS. 8-11, secondary processing of MEMS pressure sensors for implant devices can be necessary to use the sensor implant device in a biological environment and to provide functionality to relay pressure measurements to an external device that can use such readings. However, such secondary processes can be sufficiently challenging from a manufacturing perspective with respect to some packaging solutions. For example, the can package shown in FIGS. 8-11 may require substantial manual manipulations and/or single-unit operations. Furthermore, such secondary processing can introduce a relatively high failure rate and/or reduced product yield.

The present disclosure provides solutions for MEMS sensor packaging that are suitable for implantation in the heart or other anatomy of a patient. Such solutions can advantageously utilize relatively high-volume semiconductor processes to package pressure sensors. In some implementations, MEMS pressure sensor packaging may be achieved without requiring manual manipulation of the sensor device beyond semiconductor chip fabrication technologies.

Packaging processes for producing various embodiments described in the present disclosure can involve and/or provide the stabilization of wire bond connections between MEMS pressure sensor devices and associated substrates, structures, and/or circuitry, as well as insulation of such connections using, for example, silicone potting or deposition of parylene-type conformal coatings, or other polymers. Furthermore, in some embodiments, additional biocompatible coatings can be applied, including one or more layers of silicone, parylene, sputtered film (e.g., titanium film), or the like.

Pressure sensor implant devices in accordance with aspects of the present disclosure may include one or more pressure sensor devices, such as MEMS pressure sensor devices, packaged on a rigid substrate with a transduction medium applied over at least a pressure sensing diaphragm/transducer component thereof Such transduction medium may further be applied over certain electrical connections, such as wire bonds or the like connected to the sensor and/or over at least a portion of the rigid substrate/board to which the sensor device is mounted/secured. In some embodiments, electrical connection to the pressure sensor device is via a flip-chip electrical connection, or any other type of circuit board electrical connection. Therefore, it should be understood that description herein of electrical connections being covered with transduction medium may be understood to apply to any type of electrical connection (e.g., through-hole/via, bond pad, soldered connection, etc.). Where electrical connection to a MEMS pressure sensor device is via a backside of the MEMS pressure sensor, such connections may not be directly contacted by transduction medium covering, but rather the MEMS pressure sensor may be entirely covered by, transduction medium over one or more sides thereof, thereby providing insulation for the backside connection(s).

Transduction media applied over a sensor element in connection with embodiments of the present disclosure may comprise silicone, parylene (e.g., parylene C), epoxy, and/or other polymers. A transduction medium used in connection with embodiments of the present disclosure may form a conformal or non-conformal surface over the sensor device(s) and/or electrical connections covered thereby. With respect to conformal layers of transduction medium applied over sensor devices, such layers/material may conform/follow, at least in part, the outline, footprint, and/or form of the sensor device(s) and/or electrical connections thereto, as well as one or more other packaging components or electronics.

Embodiments of the present disclosure may further include a biocompatibility layer applied over the transduction medium, wherein the biocompatibility layer may advantageously provide a moisture barrier for the implant device. The term “layer” is used herein according to its broad and ordinary meaning and may refer to a thickness of material or materials covering an area. As used herein, a “layer,” such as a biocompatibility layer, may comprise a plurality of individual sub-layers that collectively provide a thickness of material( )that perform a particular function, such as providing a moisture barrier, providing a relatively inert, biocompatible interface between a structure or material and an environment or other structure or material, or the like. That is, in some implementations, a layer, such as a biocompatibility layer, may include a plurality of stacked layers of different materials or compositions. For example, biocompatibility layers in accordance with the present disclosure may include one or more layers of metal film, such as sputtered titanium film, as well as one or more layers of polymer, such as silicone or parylene. In some embodiments, an oxide layer is formed over a biocompatibility layer, wherein an organic film, such as polyethylene glycol, or other type of organic film (e.g., non-chain organic acid, protein, carbohydrate, etc.) is bonded (e.g., covalently bonded) to the surface oxide layer.

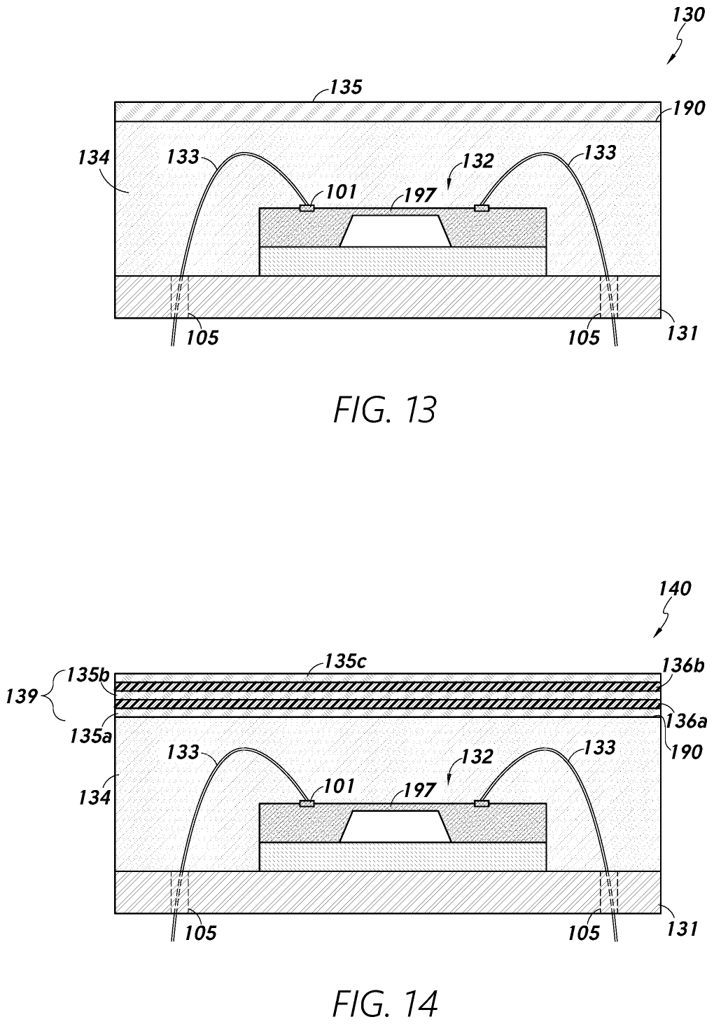

FIG. 13 shows a side view of a packaged pressure sensor device 130 in accordance with one or more embodiments of the present disclosure. The packaged pressure sensor 130 includes a MEMS pressure sensor device 132 that is disposed on a packaging substrate 131, which may comprise a circuit board, a metal sheet or base, or other structure. The substrate 131 may advantageously be rigid in some embodiments. Electrical connections to the sensor device 132 may be made through one or more apertures/through-holes 105 in the substrate 131. For example, one or more bondwires 133 or other electrical connectors may be passed through the through-holes 105 to provide electrical contact with the pressure sensor device 132. Although the through-holes 105 are shown as being positioned in the substrate 131 laterally to the side(s) of the sensor device 132, in some embodiments, the through-hole(s) 105 may be disposed/positioned directly beneath the sensor device 132, wherein electrical connections may contact the sensor device 132 on an underside thereof, or wire bonds or other contacts may be routed underneath the sensor device 132.

A transduction layer/medium 134 may be applied over the MEMS pressure sensor 132, as well as over one or more of the electrical connections 133 (e.g., wire wires, bond pads, etc.) between the sensor 132 and the packaging substrate 131 and/or other component(s). The transduction medium/layer 134 may comprise silicone, parylene, epoxy, or another polymer. The transduction layer 134 may be applied using a spin-coating process, for example, which may produce a silicone (or other material) potting over the sensor 132. In some implementations, the transduction (e.g., silicone) layer/medium is cured after application thereof to further solidify the medium. The transduction medium 134 may serve to protect, stabilize, and/or insulate the pressure sensor device 132 and/or wire bonds 133 connected to the sensor device 132. The transduction medium/layer 134 may further be applied over and/or onto at least a portion of the base substrate 131 around the sensor 132 and/or connections 133. In some implementations, the transduction medium/Layer 134 may be applied over the pressure sensor 132 and/or connections 133 in liquid form and subjected to a curing process to at least partially harden/solidify the medium to prevent runoff thereof. That is, the transduction medium 134 may not need to be contained in a sealed can package in some embodiments, wherein the transduction medium 134 has sufficient solidity to hold its form without necessary support from side walls, covers, or the like and/or regardless of orientation.

As referenced above, it may generally be considered undesirable to apply materials to an active membrane/diaphragm component of a MEMS pressure sensor device due to the potential to obstruct or interfere with the deflection of the membrane/diaphragm, thereby impairing pressure-sensing functionality of the sensor and/or reducing the sensitivity, thereof. Therefore, the transduction layer 134 of FIG. 13 may advantageously have characteristics that allow mechanical pressure to be transferred/translated therethrough, such that the pressure and/or deformation experienced at the surface 190 of the transduction layer 134 results in commensurate deflection of the sensor membrane/diaphragm 197 with little or no loss of sensitivity.

The transduction layer 134 is further covered or coated by/with a biocompatibility layer 135, which may comprise one or more layers of titanium or other material that is relatively inert when implanted in a cardiac chamber or other target location of the body and/or resistant or impermeable to moisture. In some embodiments, the biocompatibility layer 135 comprises titanium film, which may be applied using a sputtering process, or through any other application process.

In some implementations, the potting/application process for applying the silicone or other polymer transduction medium 134 can produce a relatively non-conformal upper surface 190. That is, the upper service 190 of the transduction medium 134 may be relatively flat/planar and/or not substantially conforming to the shape and/or form of the components onto which it is applied, such as the sensor 132 and/or connections 133. In some implementations, the surface 190 of the transduction layer 134 may be at least partially concave or convex over the sensor device 132, wherein such concavity may be a result of surface tension and/or other characteristics of the material of the transduction layer/medium 134.

The material of the transduction medium/layer 134 may comprise a relatively flexible polymer. Although silicone, epoxy, parylene, and the like are explicitly referenced herein, it should be understood that other types of relatively flexible/soft polymer materials may be used in connection with embodiments of the present disclosure. For example, low-durometer epoxies and/or similar polymer materials may be used that have characteristics that allow for the translation of mechanical pressure therethrough.

As described, the biocompatibility layer 135 may comprise titanium in some embodiments. However, it should be understood that the biocompatibility layer 135 may alternatively or additionally comprise one or more layers of silicone, parylene, and/or other type(s) of sputtered films.

As referenced throughout the present disclosure, an example material that may be used as a transduction medium and/or biocompatibility layer in accordance with various embodiments of the present disclosure is parylene. “Parylene,” as used herein, can refer to polymers including, for example, para-benzenediyl rings (e.g., C₆H₄) connected by 1,2-ethanediyl bridges (e.g., CH₂—CH₂). In some embodiments, parylene can be obtained by polymerization of para-xylylene (e.g., H₂C—C₆H₄—CH₂). The term “parylene,” as used herein, may also refer to polymers with similar structures, wherein some hydrogen atoms are replaced by other functional groups. For example, such variants can be identified by certain letter-number codes, such as “parylene C” and “parylene AF-4.” Coatings of parylene can be applied to embodiments of the present disclosure to provide electrical insulation, moisture barriers, or protection against corrosion and/or chemical damage for relatively long-term implantation in the heart or other anatomy. Parylene coatings/layers in pressure sensor packagings of the present disclosure can further serve to reduce friction and/or prevent adverse reactions to the implanted devices. Parylene films and/or layers applied as part of pressure sensor packagings disclosed herein (e.g., as part of a biocompatibility layer) may be applied using any suitable or desirable process, including chemical vapor deposition, For example, such depositions may be implemented in an atmosphere of the monomer para-xylylene.

Although a single stripe/layer of biocompatibility material 135 is shown in FIG. 13, it should be understood that biocompatibility layers disclosed herein in connection with various embodiments of the present disclosure may have any suitable or desirable number of layers stacked together. For example, in some implementations, biocompatibility layers of the present disclosure comprise alternating layers of metal and polymer films. FIG. 14 shows a side view of an example configuration of the sensor packaging shown in FIG. 13, including a biocompatibility layer 139 that includes one or more metal film layers 135 and one or more interleaved polymer layers 136, such as parylene or the like. Although the metal film layer 135a is shown as being directly deposited on the transduction medium layer 134, in some implementations, a layer of parylene or other polymer film may be directly applied to the top surface 190 of the transduction layer 134, wherein a metal film layer (e.g., sputtered titanium film) is applied thereon.

Although FIG. 14 shows three metal film layers 135 and two polymer film layers 136 in the biocompatibility layer 139, it should be understood that biocompatibility coatings/layers in accordance with aspects of the present disclosure may comprise any suitable or desirable number of metal film layers and polymer film layers. Furthermore, although alternating metal film and polymer film layers are shown, in some implementations, multiple polymer film layers of different types of polymer and/or multiple metal film layers of different types of metals may be directly stacked on one another. For example, the biocompatibility layer 139 may comprise alternating groups of layers of metal and polymer films. References herein to layers of a biocompatibility layer may be understood to refer to sublayers of a biocompatibility layer comprising a stack of sublayers stacked or disposed on one another.

Although an odd number of layers in the biocompatibility layer 139 is shown, wherein metal film 135 is present as the top and bottom layers of the stack 139, it should be understood that biocompatibility layers/features in accordance with aspects of the present disclosure may include an even number of layers of alternating metal and polymer film with one type of file (e.g., metal or polymer) as the bottom layer and the other type metal or polymer) as the top layer. In some embodiments, the biocompatibility layer 139 consists of an odd number of layers of film, wherein the top and bottom layers comprise polymer film. With respect to biocompatibility layers consisting of an odd number of film sublayers, any number of sublayers may be implemented, including but not limited to 3, 5, 7, 9, 11, 13, or 15 sublayers. With respect to biocompatibility layers consisting of an even number of film sublayers, any number of sublayers may be implemented, including but not limited to 2, 4, 6, 8, 10, 12, 14, or 16 sublayers.

Any embodiments of the present disclosure may include biocompatibility layers having alternating layers of metal and polymer films as shown in FIG. 14 and or described in connection therewith. Furthermore, the sublayers of such biocompatibility layers biocompatibility layer 139) can be any suitable or desirable thickness. Furthermore, the polymer film sublayers and metal film sublayers can have the same thickness, or the polymer film sublayers may have a different thickness than the metal film sublayers. The thickness of the individual sublayers and the overall thickness of the biocompatibility layer are advantageously designed to provide desirable flexibility for the efficient translation of pressure therethrough (i.e., pressure transparency), while providing hermetic sealing. For example, polymer film and/or metal film sublayers of a biocompatibility layer can have a thickness of approximately 1 μm or less. In some embodiments, the polymer film and/or metal film sublayers are less than or equal to about 100 nm. In some embodiments, the sum total of the thicknesses of the sublayers of the biocompatibility layer 139 produces a total thickness of the biocompatibility layer 139 of approximately 10 μm or less.

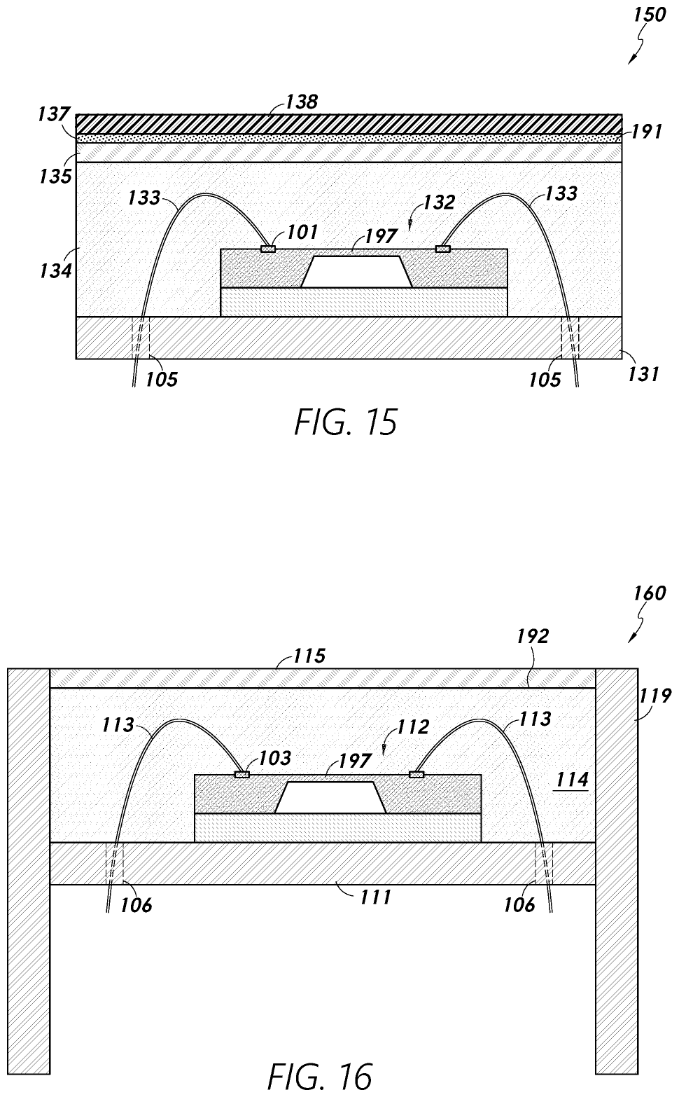

FIG. 15 shows another example configuration of the packaged pressure sensor of FIG. 13, wherein the biocompatibility layer 135, which may comprise a layer of sputtered titanium film or the like, has an oxide layer 137 formed on an upper surface 191 thereof. Further processing of an oxide-containing surface of the biocompatibility layer applied over the MEMS pressure sensor 132 may be implemented for various purposes, as described in detail below. For example, as with embodiments described above, the biocompatibility layer (e.g., sputtered titanium film) 135 is applied over the MEMS pressure sensor 132 and an intervening transduction layer 134. A surface oxide layer 137 is formed on the surface 191 of the biocompatibility layer 135, and an additional organic film layer 138 is chemically bonded to the surface oxide 137 to provide enhanced biocompatibility characteristics.

Formation of the oxide layer 137 and organic film layer 138 may require certain further processing of the biocompatibility layer 135 and/or other oxide-containing surfaces. The organic film layer 138 may be bonded to the oxide layer 137 using any suitable or desirable chemical bonding process/approach to attach the organic film 138 to the oxide layer 137. In some embodiments, the organic film layer 138 may be covalently bonded to the oxide layer 137, which may provide robust bonding characteristics and/or improved biocompatibility features. The organic film layer 138 may comprise any suitable or desirable organic material, such as one or more applications/layers of polyethylene glycol (PEG), long-chain organic acid(s), protein(s), carbohydrate(s), and/or the like.

In accordance with some embodiments of the present disclosure, MEMS pressure sensor devices may be processed to be covered and/or insulated by one or more transduction layers and/or biocompatibility layers, wherein the pressure sensor device is disposed within a can package including one or more sidewalls forming a can or cup in which the sensor device can be placed. FIG. 16 shows a pressure sensor package 160 including one or more transduction layers 114 and one or more biocompatibility layers 115 applied or deposited over a MEMS pressure sensor 112 within a can package having one or more side walls 119. The sensor device 112 is disposed/nested in a compartment formed by the base 111 and the side wall(s) 119, as shown. In some embodiments, the side walls 119 surround the sensor device 112 around a circular radius.

In the embodiment of FIG. 16, as with certain other embodiments disclosed above, the transduction medium/layer 114 is applied over the sensor device 112. The transduction medium/layer 114 may comprise silicone, parylene, epoxy, or other at least partially flexible polymer, and may be deposited (e.g., using spin-coating or other application process) within the side walls 119 of the can package 110. As shown, the transduction medium/layer 114 may be generally non-conformal, such that the top surface 192 thereof does not follow the form of the pressure sensor device 112 and/or connections 113 over which the transduction layer 114 is applied.

A biocompatibility layer 115 may further be applied over the transduction layer 114. The biocompatibility layer 115, as with certain other embodiments disclosed herein, can comprise titanium film or other metal film, which may be applied using a sputtering process, for example. Furthermore, it should be understood that the biocompatibility layer 115 may comprise multiple layers, such as alternating layers, or groups of layers, of metal and polymer films, as described in detail above with reference to FIG. 14. Therefore, aspects of FIG. 14 relating to biocompatibility layers, as well as the associated text description, should be understood to apply to the biocompatibility layer 115 in certain embodiments.

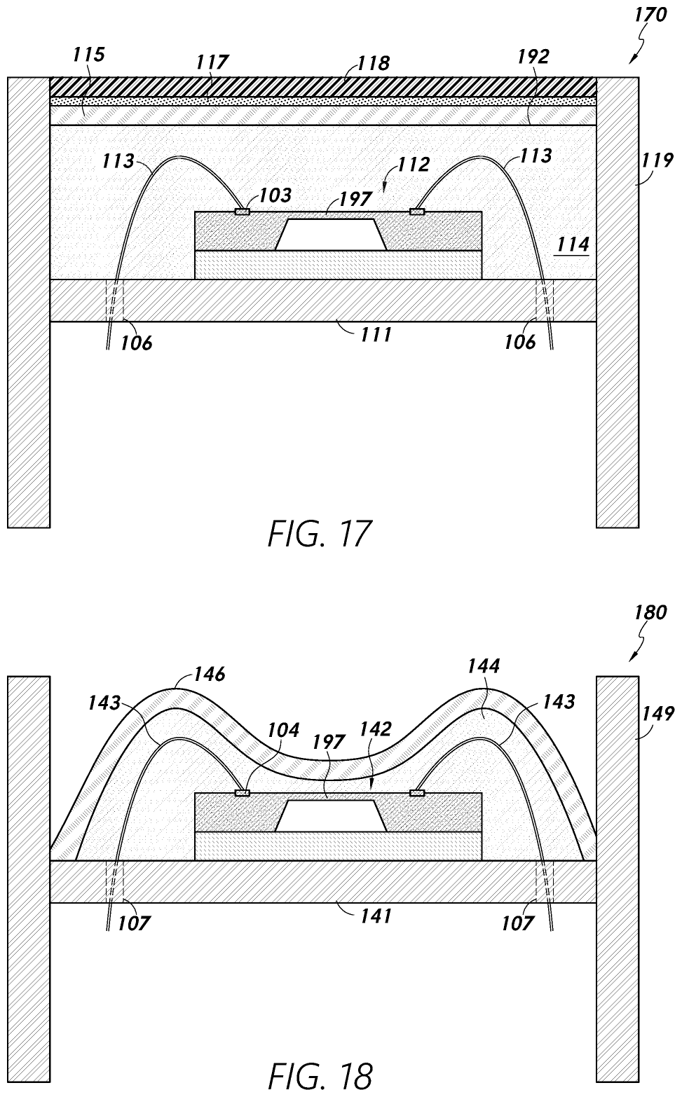

FIG. 17 shows an example configuration of the pressure sensor package shown in FIG. 16, wherein the biocompatibility layer 115 is further processed in a similar manner as described above in connection with FIG. 15. That is, in the embodiment shown in FIG. 17, the biocompatibility layer 115 has an oxide layer 117 formed thereon, with an organic film 118 that is bonded to the oxide layer to provide enhanced biocompatibility characteristics. It should be understood that the oxide layer 117 and organic film layer 118 of FIG. 17 may be similar in various respects to the oxide and organic film layers described above in connection with FIG. 15.

FIG. 18 shows an embodiment of a pressure sensor package 140 including one or more conformal layers of transduction medium and/or biocompatibility materials. That is, whereas certain other embodiments disclosed herein include transduction and/or biocompatibility layers that are relatively flat and/or have certain concavities that do not follow or conform to the shapes/forms of the devices or components onto which they are applied, the embodiment of FIG. 18, as well as various other embodiments described below, includes layers that conform to the forms of the devices that they cover, which may provide improved sensitivity with respect to translation of mechanical forces through the transduction media, and/or improved sealing and/or moisture-barrier characteristics with respect to biocompatibility layers. Furthermore, such devices may have reduced bulkiness and/or form factors with respect to one or more dimensions, which may be advantageous for implant devices that are required to be delivered to a target implantation site through relatively small delivery systems and/or through tortuous anatomical access paths blood vessels). Use of conformal transduction and/or biocompatibility layers may be enabled in accordance with embodiments of the present disclosure due to the relatively solid states of the respective layers, unlike other solutions implementing liquid transduction mediums, which do not allow for conformal surfaces in most cases.

In the embodiment of FIG. 18, the pressure sensor device 142 is covered first with a conformal insulating and transducing medium layer 144. The transduction medium 144 may comprise, for example, parylene, silicone, epoxy, or other polymer deposition, and may be deposited using chemical vapor deposition or other process(es). The transduction medium 144 may be applied over the pressure sensor 142, as well as over one or more electrical connections 143, such as wire bonds or the like, as shown.

The biocompatibility layer 146 may further be applied over the transduction medium layer 144. The biocompatibility layer 146 may comprise one or more layers of metal film (e.g., sputtered titanium film) and/or polymer film (e.g., parylene). In some implementations, the biocompatibility layer 146 may be deposited at least in part using a sputtered deposition process, for example. The biocompatibility layer 146 may similarly be conformal to the surface onto which is deposited. For example, the surface of the biocompatibility layer 46 may conform to the form or shape of the sensor device 142, connections 143, and/or transduction medium layer 144.

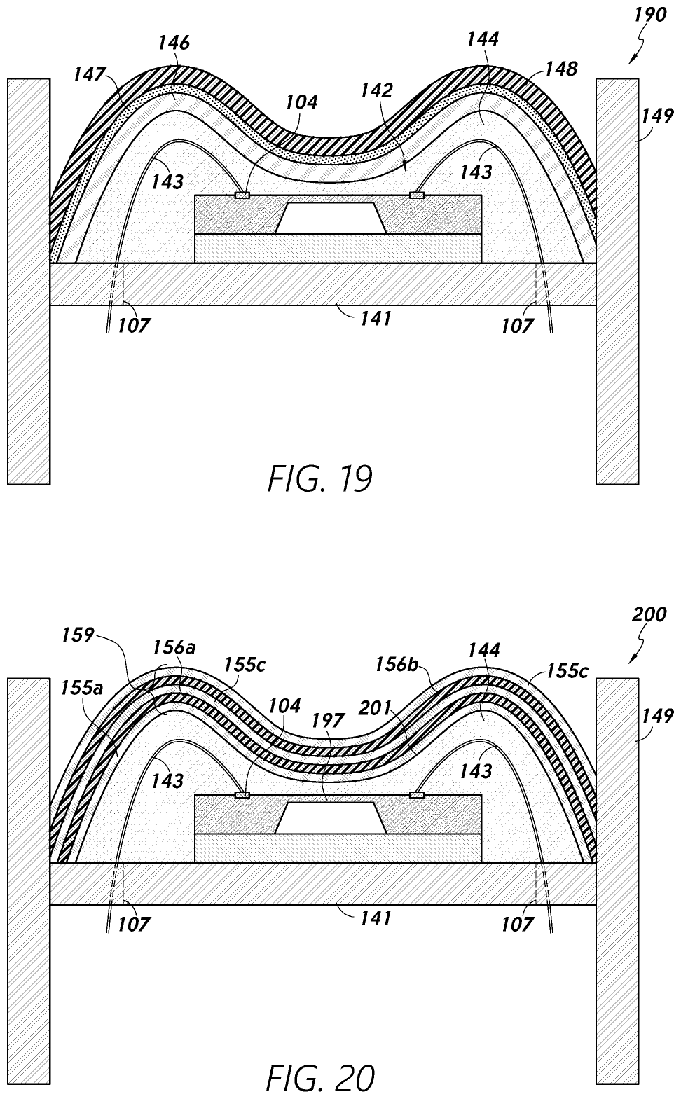

FIG. 19 shows an example configuration of the pressure sensor package shown in FIG. 18, wherein the biocompatibility layer 146 is further processed in a similar manner as described above in connection with FIG. 15. That is, in the embodiment shown in FIG. 19, the biocompatibility layer 146 has an oxide layer 147 formed thereon, with an organic film 148 that is bonded to the oxide layer to provide enhanced biocompatibility characteristics. It should be understood that the oxide layer 147 and organic film layer 148 of FIG. 19 may be similar in various respects to the oxide and organic film layers described above in connection with FIG. 15.

FIG. 20 shows a side view of an example configuration of the sensor packaging shown in FIG. 18, including a biocompatibility layer 159 that includes one or more metal film layers 155 and one or more interleaved polymer layers 156, such as parylene or the like. Although the metal film layer 155a is shown as being directly deposited on the transduction medium layer 144, in some implementations, a layer of parylene or other polymer film may be directly applied to the top surface 201 of the transduction layer 144, wherein a metal film layer (e.g., sputtered titanium film) is applied thereon.

Although FIG. 20 shows three metal film layers 155 and two polymer film layers 156 in the biocompatibility layer 159, it should be understood that biocompatibility coatings/layers in accordance with aspects of the present disclosure may comprise any suitable or desirable number of metal film layers and polymer film layers. Furthermore, although alternating metal film and polymer film layers are shown, in some implementations, multiple polymer film layers of different types of polymer and/or multiple metal film layers of different types of metals may be directly stacked on one another. For example, the biocompatibility layer 159 may comprise alternating groups of layers of metal and polymer films.

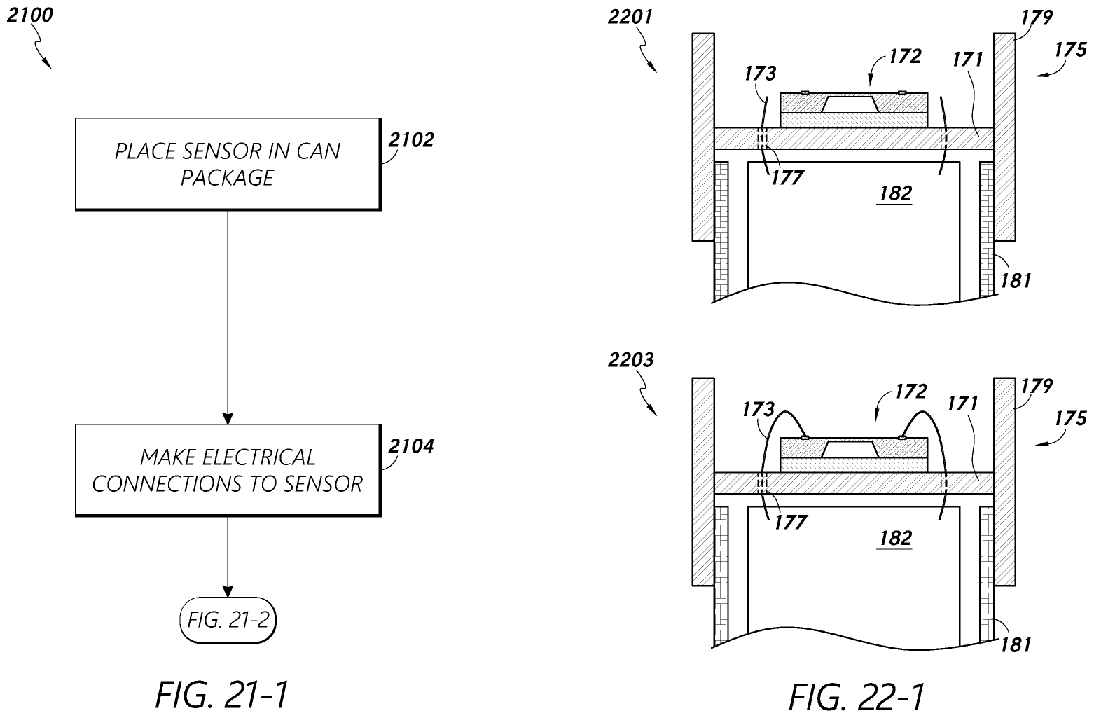

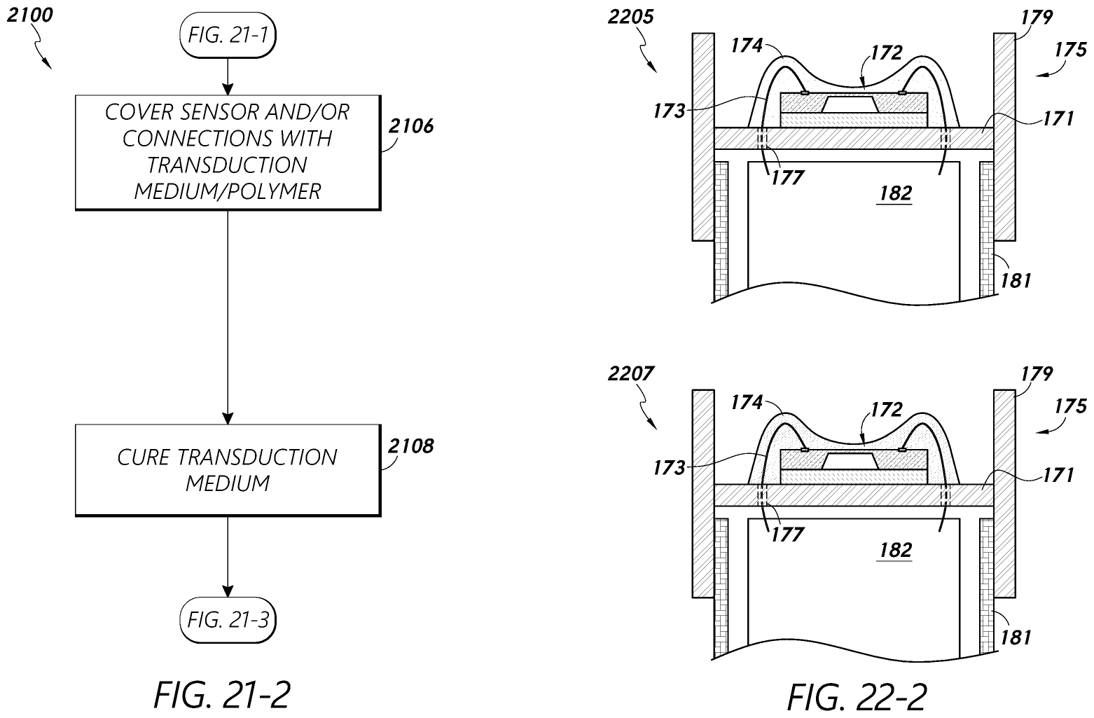

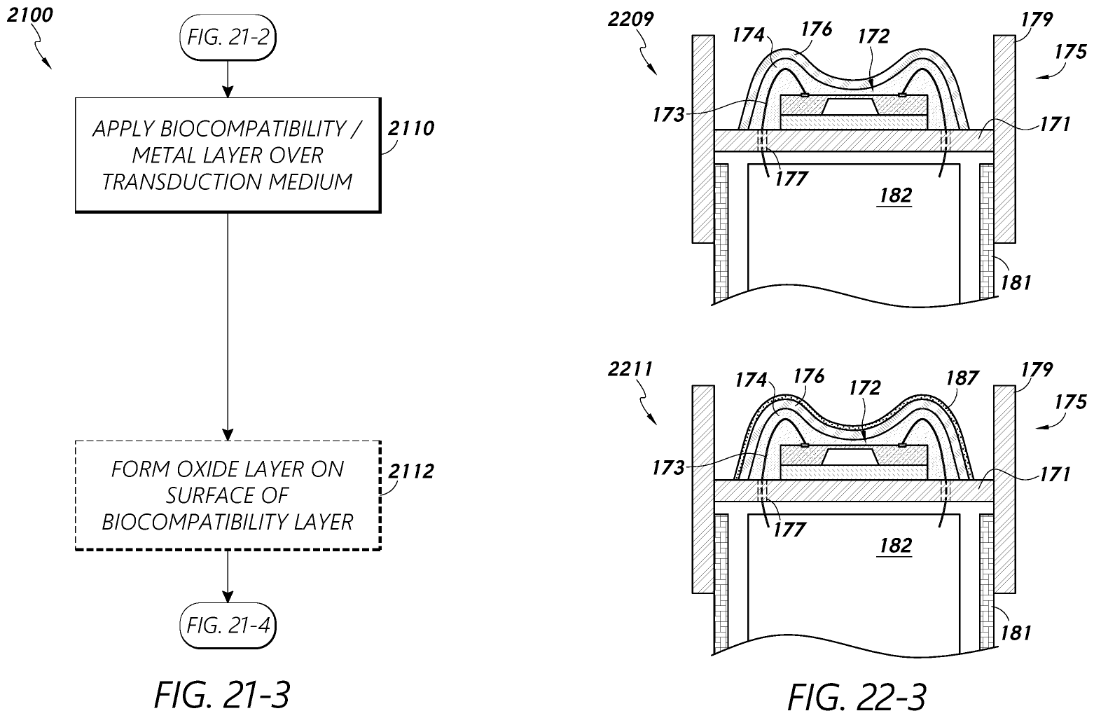

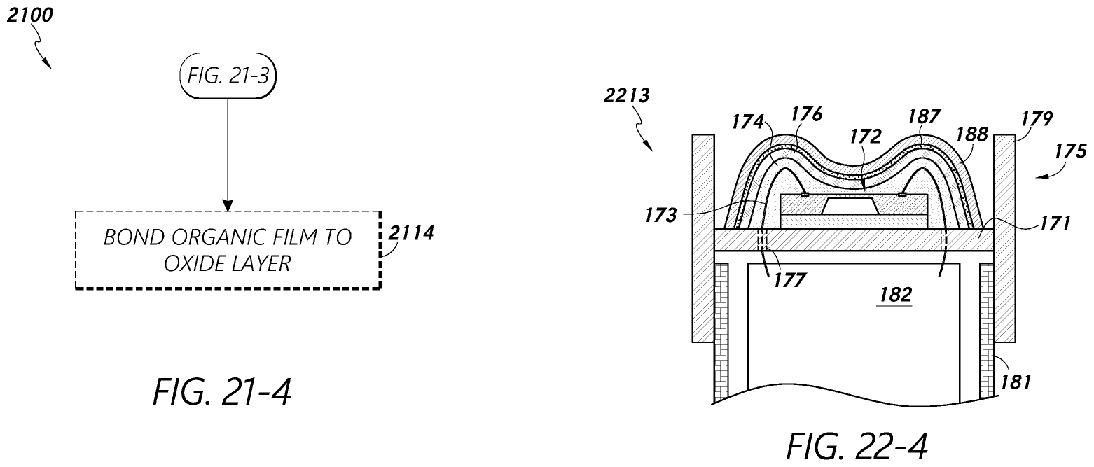

FIGS. 21-1-21-4 are a flow diagram illustrating a process 2100 for packaging a sensor implant device in accordance with some embodiments of the present disclosure. FIGS. 22-1-22-4 provide images of pressure sensor packaging corresponding to operations of the process of FIGS. 21-1-21-4 according to one or more embodiments.

At block 2102, the process 2000 involves placing a MEMS pressure sensor 172 in a can package 175. Image 2201 shows the can package 175 with the MEMS pressure sensor 172 disposed therein. The can package 175 is shown as having side walls 179. In some embodiments, the can package 175 does not include such sidewalls. Rather, the pressure sensor 172 may be placed on a base 171 that is not associated with sidewalls. In some embodiments, the can package 175 is physically coupled to a tubular housing 181, or other structure, which may house certain electronics/circuitry associated with the sensor 172.

At block 2104, the process 2100 involves making electrical connections to the sensor device 172 through the base 171 of the can package 175. For example, in some embodiments, bondwires 173 may be electrically coupled to a circuit board 182 through apertures, through-holes, or other features 177 in the base 171. At block 2106, the process 2100 involves covering the sensor device 172 and/or the electrical connections (e.g., bondwires) 173 with transduction medium 174, which may comprise one or more layers of parylene, silicone, epoxy, or the like.

At block 2108, the process 2100 may involve curing the transduction medium 174. For example, in some embodiments, the transduction medium 174 may be applied in an at least partially liquid state, wherein curing in connection with the operation(s) associated with block 2108 can serve to solidify the transduction medium. At block 2110, the process 2100 involves applying a biocompatibility layer 176 over the transduction medium 174, which may involve applying one or more layers of metal and/or polymer film, as described in detail herein. At block 2112, the process 2100 involves forming an oxide layer 187 on a surface of the biocompatibility layer 176. At block 2114, the process 2100 involves bonding an organic film layer 188 to the oxide layer 187.

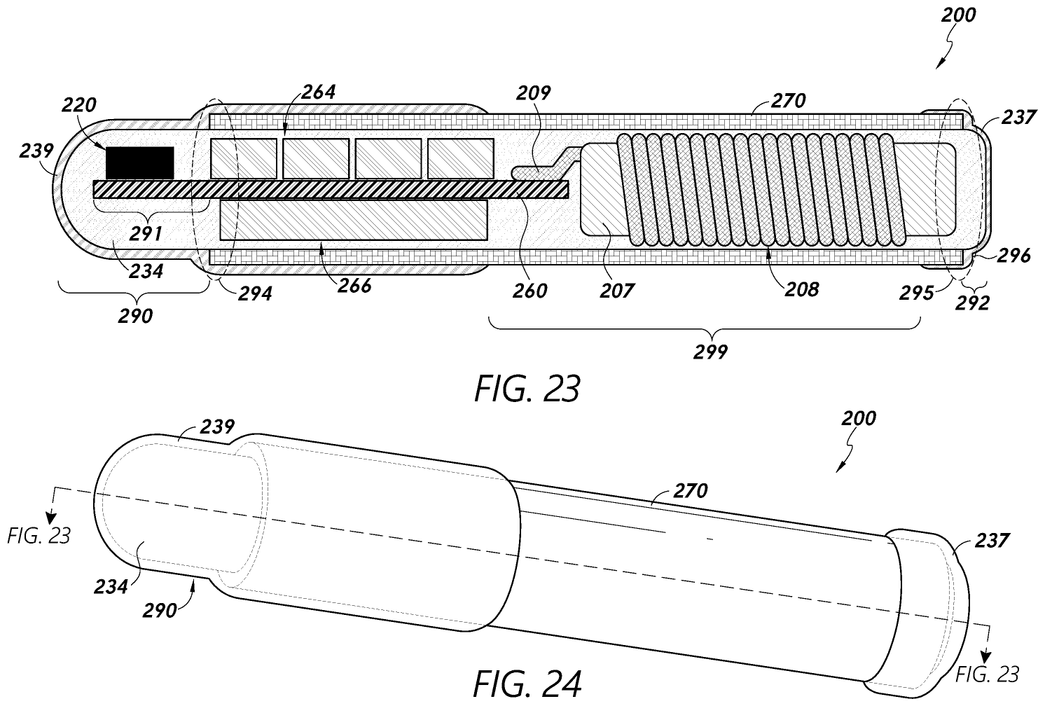

Various embodiments are described above relating to pressure sensor implant devices in which one or more MEMS pressure sensor devices are packaged in a can package, such as a metal can or the like. In some embodiments, a pressure sensor implant device may include one or more MEMS pressure sensor devices disposed on a circuit board or other non-can substrate or structure. FIG. 23 is a cross-sectional side view of a sensor implant device 200 including a MEMS pressure sensor device 220 disposed on a circuit board component 260, wherein the sensor device 220 is not contained within a can package.

The sensor implant device 200 includes certain electronics, including one or more passive circuitry components 264 and/or integrated circuit components 266, which may be disposed on either side of the circuit board 260. The implant device 200 further includes a wireless transmission component 208, which may comprise a coil antenna, or other telemetry feature. The sensor implant device 200 further includes a housing structure 270, which may comprise a rigid cylindrical tube or similar structure. In some embodiments, a portion 291 of the circuit board 260 may be configured and/or positioned to extend axially passed an end 294 of the housing 270, such that the sensor device 220, which may be disposed on the portion 291 of the board 260, is not covered by the housing 270.

The sensor implant device 200 may include a polymer potting 234, which may be injected or poured/flowed through the housing 270 to thereby cover the various circuitry/electrical components of the implant device 200, as shown in FIG. 23. The polymer potting 234 may serve as a transduction medium, as described in detail herein, which may be configured to transmit pressure applied thereto to the pressure diaphragm of the pressure sensor device 220. With the transduction medium 234 applied over the circuit board 260 and sensor device 220, at least a portion 290 of the transduction medium 234 may extend axially beyond the end 294 of the tube housing 270, such that a projection 290 of polymer transduction medium extends from beyond the end 294 of the housing 270. In some embodiments, the opposite end 295 of the housing 270 may likewise have a portion 292 of transduction medium projecting from an opening thereof

The sensor implant device 200 may further include one or more portions 237, 239 of biocompatibility material(s)/layer(s), which may be applied over one or more portions of the exposed transduction medium 290, 292 and/or the housing 270. For example, the biocompatibility layer may be applied over the entire external surface area of the implant device 200 or may be masked such that at least a portion 299 of the housing 270 is not covered with biocompatibility material. For example, the presence of a gap 299 in the biocompatibility layer may reduce interference with the wireless transmission to and/or from the transmission element 208 when the biocompatibility material(s) comprise electrically conductive material (e.g., titanium film) that may otherwise interfere with electromagnetic radiation and/or material that may interfere with some signal transmission for ultrasound or other some signal communication devices implemented. In a particular embodiment of FIG. 23, the biocompatibility layer is masked to effectively provide distal 239 and proximal 237 portions of biocompatibility layers, each covering a respective end of the housing 270.

The biocompatibility layer 239 (and/or 237) may comprise one or more layers of metal film and/or polymer film, as described in detail herein. That is, the biocompatibility layer 239 may have any configuration as described herein with respect to any of the embodiments of biocompatibility layers disclosed. For example, the biocompatibility layer 239 may comprise a surface oxide bonded to an organic film layer, as described with respect to certain embodiments disclosed herein. In some embodiments, the biocompatibility layer 239 comprises alternating layers of metal film and polymer film, such as sputtered titanium film and sputtered or otherwise applied layers of parylene film (e.g., parylene C).

FIG. 24 shows a perspective view of the pressure sensor implant device 200 of FIG. 23 in accordance with one or more embodiments of the present disclosure. In FIG. 24, the portions 239, 237 of the biocompatibility layer are shown applied over the respective end portions of the tubular housing 270, thereby covering the projection 290 of transduction medium covering the MEMS pressure sensor(s) 220.

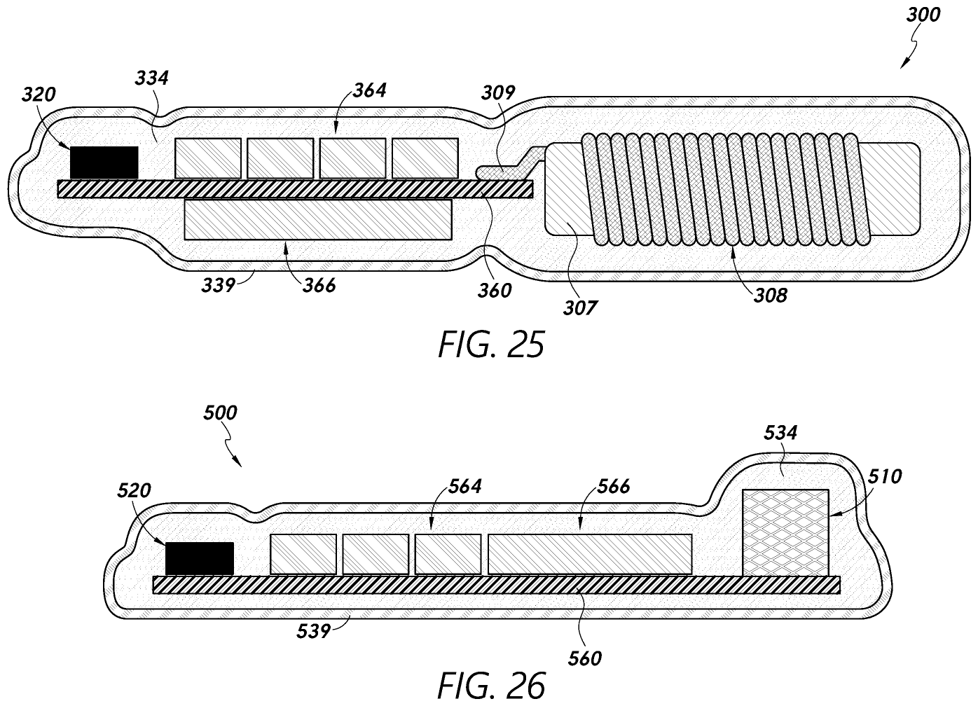

FIG. 25 shows a cross-sectional side view of a pressure sensor implant device 300 that is similar in certain respects to the pressure sensor implant device 200 described above in connection with FIGS. 23 and 24, wherein the pressure sensor implant device 300 does not include a tubular housing covering portions of the electronics of the implant device 300. For example, a conformal layer of transduction medium 334 may be applied over all of the electronics of the implant device 300, including the wireless transmission element/device 308 (e.g., coil antenna, piezoelectric resonator, etc.), circuit board 360, passive 364 and/or integrated circuit 366 chips/components, MEMS pressure sensor device(s) 320, and/or electrical connections thereto. Some or all of the outer surface of the transduction medium 334 may be covered with biocompatibility material 339, as described herein. That is, the biocompatibility material 339 may have any configuration as described herein with respect to biocompatibility layers/materials described in connection with any of the embodiments disclosed. For example, the biocompatibility layer 339 may comprise alternating layers of titanium film and parylene, or any other configuration disclosed herein. In some embodiments, the biocompatibility layer 339 comprises a surface oxide layer formed on a metal film (e.g. sputtered titanium film), wherein an organic film layer is bonded (e.g., covalently bonded) to the surface oxide layer.

The implant device 300 may be coated in the transduction medium 334 at least in part by disposing the assembly in a cavity/mold and flowing/potting silicone or other polymer over the components thereof to coat the electronics as shown. In some implementations, the biocompatibility layer 339 includes a coating of parylene or other moisture-resistant polymer over the entire outer area/surface of the device 300, wherein certain portions, such as portions not covering the wireless transmission component 308, are coated with titanium film or other metal/conductive film using masking or other technique/process, whereas window(s) in the biocompatibility layer are masked to allow for wireless transmission. Although the transduction medium 334 and biocompatibility layer 339 are illustrated as being conformal with respect to the topology of the electronics on the board 360, in some implementations, the transduction medium 334 may comprise silicone potting or other similar polymer potting that is nonconformal. For example, the sensor implant device 300 may have a rectangular prism form factor.

FIG. 26 shows a side view of a sensor implant device 500 including a MEMS pressure sensor device 520 and an electroacoustic transducer device 510, such as a piezoelectric resonator/transducer configured to convert electrical charge into acoustic/pressure-based signals for wireless data and/or energy transmitting and/or receiving. That is, the embodiment of FIG. 26 may be similar in various respects to the sensor implant device 300 of FIG. 25, with exception of the wireless transmission device/element 510 being electroacoustic device rather than a wire coil antenna.

The implant device 500 may be coated in transduction medium 534 at least in part by disposing the assembly in a cavity/mold and flowing/potting silicone or other polymer over the components thereof to coat the electronics as shown. In some implementations, a biocompatibility layer 539 is applied over at least some of the transduction medium 534 and includes a coating of parylene or other moisture-resistant polymer. In some embodiments, the biocompatibility layer/coating 539 is applied over the entire outer area/surface of the device 500, wherein certain portions, such as portions not covering the wireless transmission component 510, are coated with titanium film or other metal/conductive film using masking or other technique/process, whereas window(s) in the biocompatibility layer are masked to allow for wireless transmission. Although the transduction medium 534 and biocompatibility layer 539 are illustrated as being conformal with respect to the topology of the electronics on the board 560, in some implementations, the transduction medium 534 may comprise silicone potting or other similar polymer potting that is nonconformal. For example, the sensor implant device 500 may have a rectangular prism form factor.

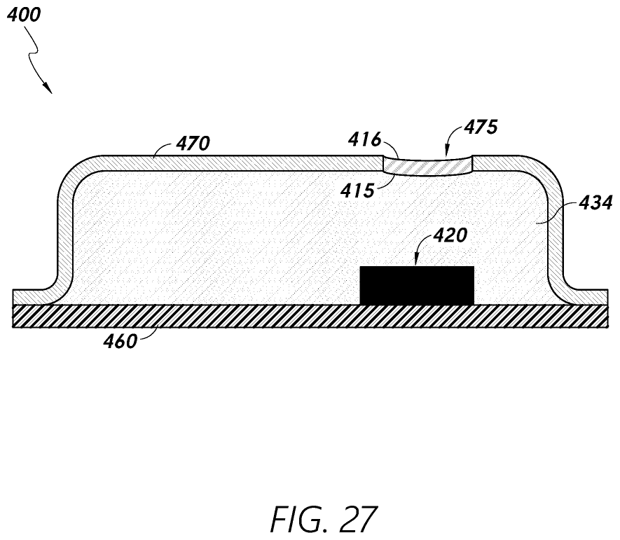

FIG. 27 shows a side cross-sectional view of a pressure sensor implant device 400 including a MEMS pressure sensor 420 disposed on a substrate 460, which may comprise any suitable or desirable at last partially rigid material, such as plastic, glass, metal, or other material. The implant device 400 further comprises a cover/housing 470, which is disposed over the pressure sensor device 420.

The cover/housing 470 may comprise an aperture 475, which may be situated/positioned at least partially over the pressure sensor device 420, such that external pressure conditions may be measured by the pressure sensor device 420 through the aperture 475. In some embodiments, the cavity within the cover 470 may be filled at least partially with transduction medium 434, which may be any type of transduction medium disclosed herein. For example, silicone, parylene, epoxy, or other relatively soft solid polymer may be used. Solid polymers may be preferable to liquid polymers due to the presence of the aperture 475, which may otherwise allow for out-flowing of a liquid transduction medium, such as silicone oil or the like.

In some implementations, the aperture 475 may be filled with a biocompatibility layer 416, which may have characteristics of any of the biocompatibility layers disclosed herein. For example, the aperture 475 may have a biocompatibility layer 416 applied on a surface 415 of the transduction medium 434 that is exposed through the aperture 475. The biocompatibility layer 416 may have any suitable or desirable number of layers, such as alternating layers of metal film and polymer film, such sputtered titanium film and/or parylene film. In some embodiments, the biocompatibility layer 416 includes a metal film with a surface oxide formed thereon, wherein an organic film layer is bonded to the surface oxide, as described in detail herein.

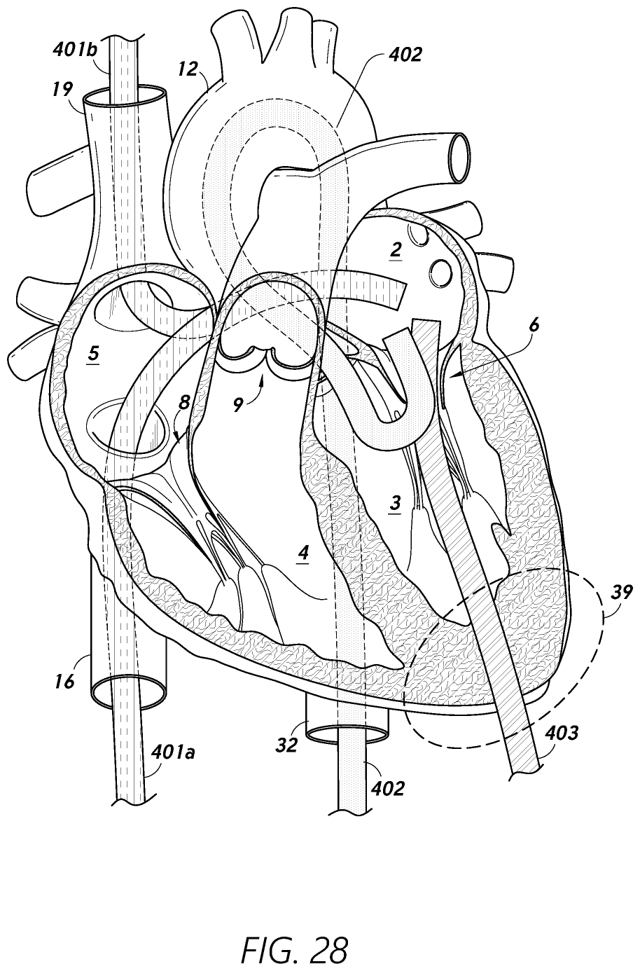

Packaged sensor implant devices in accordance with one or more embodiments of the present disclosure may be advanced to the left atrium, atrial septum, and/or any other chamber or vessel of the heart using any suitable or desirable procedure. For example, although access to various chambers/vessels of the heart is illustrated and described in connection with certain embodiments as being via the right atrium and/or inferior vena cavae, such as through a transfemoral or other transcatheter procedure, other access paths/methods may be implemented in accordance with embodiments of the present disclosure, as described/shown in connection with FIG. 28. For example, FIG. 28 illustrates various access paths through which access to the left ventricle may be achieved, including transseptal access 401a, 401b, which may be made through the inferior vena cava 16 or superior vena cava 28, as respectively shown, and from the right atrium 5, through the septal wall (not shown) and into the left atrium 2. For transaortic access 402, a delivery catheter may be passed through the descending aorta, aortic arch 12, ascending aorta, and aortic valve 7, and into the left atrium 2 through the mitral valve 6. For transapical access 403, access may be made directly through the apex of the heart into the left ventricle 3, and into the left atrium 2 through the mitral valve 6. Other access paths are also possible beyond those shown in FIG. 28.

### CERTAIN EXAMPLES

1. An implantable sensor device comprising:
a sensor-support substrate;
a microelectromechanical systems (MEMS) pressure sensor device mounted to the sensor-support substrate;
a transduction medium applied over the pressure sensor device; and
a biocompatibility layer applied over the transduction medium.

2. The implantable sensor device of example 1, further comprising one or more bondwires electrically coupled to the pressure sensor device.

3. The implantable sensor device of example 2, wherein the transduction medium covers at least a portion of the one or more bondwires.

4. The implantable sensor device of example 2 or example 3, wherein the sensor-support substrate includes one or more through-holes through which at least one of the one or more bondwires pass to a backside of the sensor-support substrate.

5. The implantable sensor device of any of examples 1 through 4, wherein the transduction medium comprises parylene.

6. The implantable sensor device of any of examples 1 through 5, wherein the transduction medium comprises silicone.

7. The implantable sensor device of any of examples 1 through 6, wherein the transduction medium comprises epoxy.

8. The implantable sensor device of any of examples 1 through 7, wherein the transduction medium has a non-conformal top surface.

9. The implantable sensor device of any of examples 1 through 8, wherein the transduction medium has a conformal surface conforming to a form of the pressure sensor device.

10. The implantable sensor device of any of examples 1 through 9, wherein the biocompatibility layer comprises a metal film.

11. The implantable sensor device of any of examples 1 through 10, further comprising an oxide layer formed on a surface of the biocompatibility layer.

12. The implantable sensor device of example 11, further comprising an organic film bonded to the oxide layer.

13. The implantable sensor device of example 12, wherein the organic film is covalently bonded to the oxide layer.

14. The implantable sensor device of example 12 or example 13, wherein the organic film comprises at least one of polyethylene glycol, a long-chain organic acid, a protein, or a carbohydrate,

15. The implantable sensor device of any of examples 1 through 14, wherein the sensor-support substrate comprises metal.

16. The implantable sensor device of any of examples 1 through 15, wherein the pressure sensor device, the transduction medium, and/or biocompatibility layer are disposed at least partially within sidewalls that are mechanically coupled to the sensor-support substrate.

17. A method of packaging a pressure sensor device, the method comprising:
providing a microelectromechanical systems (MEMS) pressure sensor device mounted to a sensor-support substrate;
applying a transduction medium over the pressure sensor device; and.
applying a biocompatibility layer over the transduction medium.

18. The method of example 17, wherein said applying the transduction medium comprises covering the pressure sensor device and at least a portion of one or more bondwires electrically coupled to the pressure sensor device with the transduction medium.

19. The method of example 17 or example 18, wherein the transduction medium comprises parylene.

20. The method of any of examples 17 through 19, wherein the transduction medium comprises silicone.

21. The method of examples 17 through 20, wherein the transduction medium comprises epoxy.

22. The method of examples 17 through 21, wherein e transduction medium has a non-conformal top surface.

23. The method of examples 17 through 22. wherein said applying the transduction medium comprises forming a conformal layer of the transduction medium over the pressure sensor device and at least a portion of the sensor-support substrate.

24. The method of examples 17 through 23; wherein said applying the biocompatibility layer comprises sputtering a titanium film onto the transduction medium.

25. The method of examples 17 through 24, further comprising forming an oxide layer on a surface of the biocompatibility layer.

26. The method of example 25, further comprising bonding an organic film to the oxide layer.

27. A pressure sensor assembly comprising:
a metal can structure including a base and one or more sidewalls;
a microelectromechanical systems (MEMS) pressure sensor device mounted to the base of the metal can structure;
a printed circuit board electrically coupled to the pressure sensor device via one or more through-holes in the base of the metal can structure;
a coil antenna electrically coupled to the printed circuit board;
a rigid tube encapsulating at least a portion of the printed circuit board and the coil antenna, the rigid tube being mechanically secured to the metal can structure;
a transduction medium applied over the pressure sensor device within the one or more sidewalk of the metal can structure; and
a biocompatibility layer applied over the transduction medium.

28. The pressure sensor assembly of example 27, wherein the transduction medium comprises parylene.

29. The pressure sensor assembly of example 27 or example 28, wherein the transduction medium comprises silicone.

30. The pressure sensor assembly of any of examples 27 through 29, wherein the transduction medium comprises epoxy.

31. The pressure sensor assembly of any of examples 27 through 30, wherein the transduction medium has a non-conformal top surface.

32. The pressure sensor assembly of any of examples 27 through 31, wherein the transduction medium has a conformal surface conforming to forms of the pressure sensor device.

33. The pressure sensor assembly of any of examples 27 through 32, wherein the biocompatibility layer comprises a metal film.

34. The pressure sensor assembly of any of examples 27 through 33, further comprising an oxide layer formed on a surface of the biocompatibility layer.

35. The pressure sensor assembly of example 34, further comprising an organic film bonded to the oxide layer.

36. A pressure sensor assembly comprising:
a printed circuit board;
a wireless transmitter electrically coupled to the printed circuit board;
a rigid tube encapsulating at least a portion of the printed circuit board and the wireless transmitter, the rigid tube having first and second ends;
a microelectromechanical systems (MEMS) pressure sensor device mounted to an end portion of the printed circuit board that extends axially beyond the first end of the rigid tube;
a transduction medium that covers the printed circuit board, the wireless transmitter, and the pressure sensor device, the transduction medium filling the rigid tube and projecting axially beyond the first end of the rigid tube over the end portion of the printed circuit board; and
a biocompatibility layer applied over the first and second ends of the rigid tube and over portions of the transduction medium associated with the first and second ends of the rigid tube, respectively.

37. The pressure sensor assembly of example 36, wherein the transduction medium comprises parylene.

38. The pressure sensor assembly of example 36 or example 37, wherein the transduction medium comprises silicone.

39. The pressure sensor assembly of any of examples 36 through 38, wherein the transduction medium comprises epoxy.

40. The pressure sensor assembly of any of examples 36 through 39, further comprising a polymer layer applied over at least a portion of the biocompatibility laver.

41. The pressure sensor assembly of any of examples 36 through 40, wherein the biocompatibility layer comprises alternating layers of polymer and metal,

42. The pressure sensor assembly of example 41, wherein the alternating layers of polymer and metal comprise at least two layers of metal and at least two layers of polymer.

43. The pressure sensor assembly of any of examples 36 through 42, further comprising an oxide layer is formed on a surface of the biocompatibility layer.

44. The pressure sensor assembly of example 43, further comprising an organic film bonded to the oxide layer.

45. An implantable sensor device comprising:
a sensor-support substrate;
a microelectromechanical systems (MEMS) pressure sensor device mounted to the sensor-support substrate;
a transduction medium applied over the pressure sensor device; and
a biocompatibility layer applied over the transduction medium, the biocompatibility layer comprising alternating sublayers of metal film and polymer film.

46. The implantable sensor device of example 45, wherein the alternating sublayers of metal film and polymer film comprise at least two sublayers of metal film and at least two sublayers of polymer film.

47. The implantable sensor device of example 46, wherein the alternating sublayers of metal film and polymer film comprise at least ten film sublayers.

48. The implantable sensor device of example 47, wherein the alternating sublayers of metal film and polymer film comprise at least twelve film sublayers.

49. The implantable sensor device of any of examples 45 through 48, at least some sublayers of the biocompatibility layer have a thickness of about 1 μm or less.

50. The implantable sensor device of any of examples 45 through 49, wherein the biocompatibility layer has a thickness of about 10 μm or less.

51. The implantable sensor device of any of examples 45 through 50, wherein bottom and top sublayers of the biocompatibility layer are metal film sublayers,

### ADDITIONAL EMBODIMENTS

Depending on the embodiment, certain acts, events, or functions of any of the processes or algorithms described herein can be performed in a different sequence, may be added, merged, or left out altogether. Thus, in certain embodiments, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain embodiments require at least one of X, at least one of Y and at least one of Z to each be present.

It should be appreciated that in the above description of embodiments, various features are sometimes grouped together in a single embodiment, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the inventions herein disclosed and claimed below should not be limited by the particular embodiments described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example embodiments belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. An implantable sensor device comprising:
a sensor-support substrate;
a microelectromechanical systems (MEMS) pressure sensor device mounted to the sensor-support substrate;
one or more bondwires electrically coupled to the pressure sensor device;
a transduction medium applied over the pressure sensor device; and
a biocompatibility layer applied over the transduction medium;
wherein the transduction medium covers at least a portion of the one or more bondwires;
wherein the sensor-support substrate includes one or more through-holes through which at least one of the one or more bondwires pass to a backside of the sensor-support substrate;
wherein the transduction medium comprises parylene, silicone, or epoxy;
wherein the transduction medium has a non-conformal top surface;
wherein the biocompatibility layer comprises a metal film, an oxide layer formed on a surface of the biocompatibility layer, and an organic film covalently bonded to the oxide layer;
wherein the organic film comprises at least one of polyethylene glycol, a long-chain organic acid, a protein, or a carbohydrate;
wherein the sensor-support substrate comprises metal; and
wherein the pressure sensor device, the transduction medium, and/or biocompatibility layer are disposed at least partially within sidewalls that are mechanically coupled to the sensor-support substrate.

2. An implantable sensor device comprising:
a sensor-support substrate;
a microelectromechanical systems (MEMS) pressure sensor device mounted to the sensor-support substrate;
a transduction medium applied over the pressure sensor device; and
a biocompatibility layer applied over the transduction medium.

3. The implantable sensor device of claim 2, further comprising one or more bondwires electrically coupled to the pressure sensor device.

4. The implantable sensor device of claim 3, wherein the transduction medium covers at least a portion of the one or more bondwires.

5. The implantable sensor device of claim 4, wherein the sensor-support substrate includes one or more through-holes through which at least one of the one or more bondwires pass to a backside of the sensor-support substrate.

6. The implantable sensor device of claim 2, wherein the transduction medium comprises parylene.

7. The implantable sensor device of claim 2, wherein the transduction medium comprises silicone.

8. The implantable sensor device of claim 2, wherein the transduction medium comprises epoxy.

9. The implantable sensor device of claim 2, wherein the transduction medium has a non-conformal top surface.

10. The implantable sensor device of claim 2, wherein the transduction medium has a conformal surface conforming to a form of the pressure sensor device.

11. The implantable sensor device of claim 2, wherein the biocompatibility layer comprises a metal film.

12. The implantable sensor device of claim 11, further comprising an oxide layer formed on a surface of the biocompatibility layer.

13. The implantable sensor device of claim 12, further comprising an organic film bonded to the oxide layer.

14. The implantable sensor device of claim 13, wherein the organic film is covalently bonded to the oxide layer.

15. The implantable sensor device of claim 14, wherein the organic film comprises at least one of polyethylene glycol, a long-chain organic acid, a protein, or a carbohydrate.

16. The implantable sensor device of claim 2, wherein the sensor-support substrate comprises metal.

17. The implantable sensor device of claim 2, wherein the pressure sensor device, the transduction medium, and/or biocompatibility layer are disposed at least partially within sidewalls that are mechanically coupled to the sensor-support substrate.

18. A method of packaging a pressure sensor device, the method comprising:
providing a microelectromechanical systems (MEMS) pressure sensor device mounted to a sensor-support substrate;
applying a transduction medium over the pressure sensor device; and
applying a biocompatibility layer over the transduction medium.

19. The method of claim 18, wherein said applying the transduction medium comprises covering the pressure sensor device and at least a portion of one or more bondwires electrically coupled to the pressure sensor device with the transduction medium.

20. The method of claim 19, wherein the transduction medium comprises silicone.
